(19) United States
(12) Patent Application Publication
(10) Pub. No.: US 2024/0199552 A1
(43) Pub. Date: Jun. 20, 2024

(54) AMIDE COMPOUND AND USE THEREOF

(71) Applicants: Metisa Biotechnology Co., Ltd, Nanning City (CN); Shenyang University of Chemical Technology, Shenyang City (CN)

(72) Inventors: Lixin ZHANG, Shenyang City (CN); Jing ZHANG, Shenyang City (CN); Hongyan PEI, Shenyang City (CN); Zhuo KANG, Shenyang City (CN)

(21) Appl. No.: 18/410,006

(22) Filed: Jan. 11, 2024

Related U.S. Application Data
(63) Continuation of application No. PCT/CN2022/104612, filed on Jul. 8, 2022.

(30) Foreign Application Priority Data
Jul. 12, 2021 (CN) 202110782785.2

(51) Int. Cl. C07D 213/82; A01N 37/46; A01N 43/40; A01P 7/04; A61K 31/167; A61K 31/277; A61K 31/455; A61P 33/14; C07C 237/42; C07C 255/57; C07D 213/84

(52) U.S. Cl. CPC C07D 213/82; A01N 37/46; A01N 43/40; A01P 7/04; A61K 31/167; A61K 31/277; A61K 31/455; A61P 33/14; C07C 237/42; C07C 255/57; C07D 213/84

(57) ABSTRACT

The present invention discloses an amide compound and a use thereof, and the structure of the compound is shown in General Formula I:

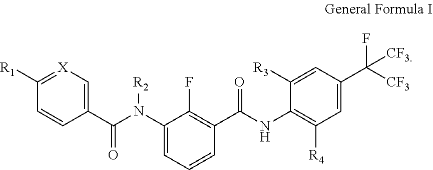

The definitions of each substituent in the formula can be found in the description; and the uses as an insecticide and an animal parasite control agent are also disclosed in the description.

### TECHNICAL FIELD

The present invention relates to a compound, particularly to a new-type amide compound and a use thereof.

### BACKGROUND ART

Patent JP2007099761A discloses the following specific compounds KC1 (CAS registry number: 934532-14-6) and KC2 (CAS registry number: 934532-15-7), which have certain insecticidal activity.

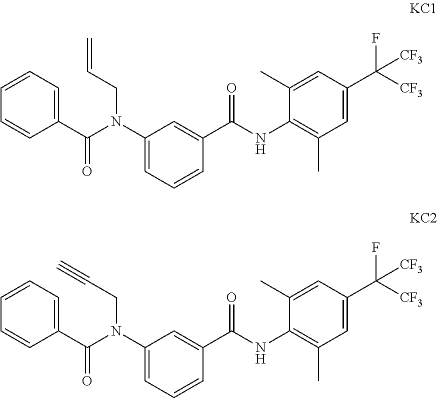 [SMILES: C=CCN(C(=O)c1ccccc1)c1cccc(C(=O)Nc2c(C)cc(C(F)(C(F)(F)F)C(F)(F)F)cc2C)c1 ; C#CCN(C(=O)c1ccccc1)c1cccc(C(=O)Nc2c(C)cc(C(F)(C(F)(F)F)C(F)(F)F)cc2C)c1]

Patent CN102119143A discloses the following compounds KC3 (compound number: 7-1574, and CAS registry number: 1207727-04-5), KC4 (compound number: 7-1577, and CAS registry number: 1207727-08-9), and KC5 (compound number: 7-1733, and CAS registry number: 1207727-07-8) with insecticidal activity. Among them, the compound KC3 has already been commercialized as an agricultural insecticide, commonly known as broflanilide in English.

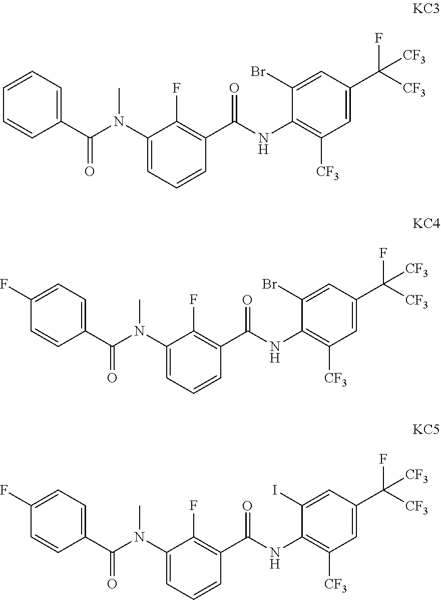 [SMILES: CN(C(=O)c1ccccc1)c1cccc(C(=O)Nc2c(Br)cc(C(F)(C(F)(F)F)C(F)(F)F)cc2C(F)(F)F)c1F ; CN(C(=O)c1ccc(F)cc1)c1cccc(C(=O)Nc2c(Br)cc(C(F)(C(F)(F)F)C(F)(F)F)cc2C(F)(F)F)c1F ; CN(C(=O)c1ccc(F)cc1)c1cccc(C(=O)Nc2c(I)cc(C(F)(C(F)(F)F)C(F)(F)F)cc2C(F)(F)F)c1F]

Patent CN102119143A also discloses the following compounds KC6 (compound number: 6-1772, and CAS registry number: 1331922-53-2), KC7 (compound number: 7-1616, and CAS registry number: 1207979-50-7), and KC8 (compound number: 7-1772, and CAS registry number: 1332266-07-5) with insecticidal activity. Among them, the compound KC6 is under development as an insecticide, commonly known as mivorilaner in English.

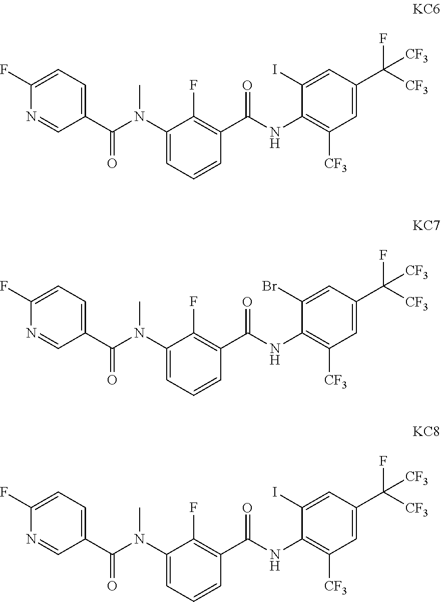 [SMILES: CN(C(=O)c1ccc(F)nc1)c1cccc(C(=O)Nc2c(I)cc(C(F)(C(F)(F)F)C(F)(F)F)cc2C(F)(F)F)c1F ; CN(C(=O)c1ccc(F)nc1)c1cccc(C(=O)Nc2c(Br)cc(C(F)(C(F)(F)F)C(F)(F)F)cc2C(F)(F)F)c1F ; CN(C(=O)c1ccc(F)nc1)c1cccc(C(=O)Nc2c(I)cc(C(F)(C(F)(F)F)C(F)(F)F)cc2C(F)(F)F)c1F]

However, the prevention and control effects of existing insecticides are still not ideal, and it is still necessary to continuously develop new, more efficient, and broad-spectrum insecticides to meet market demands.

The compound shown in General Formula I of the present invention and its insecticidal activity have not been reported yet in existing technologies.

### SUMMARY OF THE INVENTION

A purpose of the present invention is to provide an amide compound with excellent insecticidal activity. It can be used to prepare a drug for preventing and controlling pests in agriculture and other fields and a drug for controlling animal parasites in the field of veterinary drugs.

In order to achieve the purpose of the present invention, the present invention provides the following technical solutions:

An amide compound is shown in General Formula I:

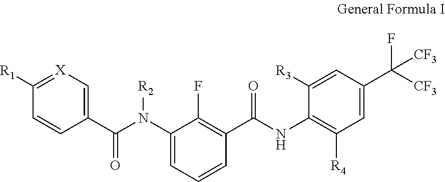

in General Formula I:
R₁ is selected from hydrogen, fluorine, cyano, trifluoromethyl or difluoromethyl;
R₂ is selected from allyl or propargyl;
R₃ selected from halogen;
R₄ is selected from halogen, C₁-C₃ haloalkyl, or C₁-C₃ haloalkoxy; and
X is selected from CH or N.

In a possible embodiment, in General Formula I,
R₁ is selected from hydrogen, fluorine, cyano, trifluoromethyl or difluoromethyl;
R₂ is selected from allyl or propargyl;
R₃ is selected from bromine or iodine;
R₄ is selected from bromine, iodine, trifluoromethyl or difluoromethoxy; and
X is selected from CH or N.

In a possible embodiment, the amide compound is selected from compounds in Table 1. The compounds in Table 1 have the structure shown in General Formula I, and R₁, X, R₂, R₃, and R₄ are shown in Table 1:

TABLE 1

| Compound | | | | | |
|---|---|---|---|---|---|
| Number | R₁ | X | R₂ | R₃ | R₄ |
| 1 | H | CH | Allyl | Br | Br |
| 2 | H | CH | Allyl | Br | I |
| 3 | H | CH | Allyl | Br | CF₃ |
| 4 | H | CH | Allyl | I | CF₃ |
| 5 | F | CH | Allyl | Br | Br |
| 6 | F | CH | Allyl | Br | I |
| 7 | F | CH | Allyl | Br | CF₃ |
| 8 | F | CH | Allyl | I | CF₃ |
| 9 | CN | CH | Allyl | Br | Br |
| 10 | CN | CH | Allyl | Br | I |
| 11 | CN | CH | Allyl | Br | CF₃ |
| 12 | CN | CH | Allyl | I | CF₃ |
| 13 | CF₃ | CH | Allyl | Br | Br |
| 14 | CF₃ | CH | Allyl | Br | I |
| 15 | CF₃ | CH | Allyl | Br | CF₃ |
| 16 | CF₃ | CH | Allyl | I | CF₃ |
| 17 | F | N | Allyl | Br | Br |
| 18 | F | N | Allyl | Br | I |
| 19 | F | N | Allyl | Br | CF₃ |
| 20 | F | N | Allyl | I | CF₃ |
| 21 | CN | N | Allyl | Br | Br |
| 22 | CN | N | Allyl | Br | I |
| 23 | CN | N | Allyl | Br | CF₃ |
| 24 | CN | N | Allyl | I | CF₃ |
| 25 | CF₃ | N | Allyl | Br | Br |
| 26 | CF₃ | N | Allyl | Br | I |
| 27 | CF₃ | N | Allyl | Br | CF₃ |
| 28 | CF₃ | N | Allyl | I | CF₃ |
| 29 | H | CH | Propargyl | Br | Br |
| 30 | H | CH | Propargyl | Br | I |
| 31 | H | CH | Propargyl | Br | CF₃ |
| 32 | H | CH | Propargyl | I | CF₃ |
| 33 | F | CH | Propargyl | Br | Br |
| 34 | F | CH | Propargyl | Br | I |
| 35 | F | CH | Propargyl | Br | CF₃ |
| 36 | F | CH | Propargyl | I | CF₃ |
| 37 | CN | CH | Propargyl | Br | Br |
| 38 | CN | CH | Propargyl | Br | I |
| 39 | CN | CH | Propargyl | Br | CF₃ |
| 40 | CN | CH | Propargyl | I | CF₃ |
| 41 | CF₃ | CH | Propargyl | Br | Br |
| 42 | CF₃ | CH | Propargyl | Br | I |
| 43 | CF₃ | CH | Propargyl | Br | CF₃ |
| 44 | CF₃ | CH | Propargyl | I | CF₃ |
| 45 | F | N | Propargyl | Br | Br |
| 46 | F | N | Propargyl | Br | I |
| 47 | F | N | Propargyl | Br | CF₃ |
| 48 | F | N | Propargyl | I | CF₃ |
| 49 | CN | N | Propargyl | Br | Br |
| 50 | CN | N | Propargyl | Br | I |
| 51 | CN | N | Propargyl | Br | CF₃ |
| 52 | CN | N | Propargyl | I | CF₃ |
| 53 | CF₃ | N | Propargyl | Br | Br |
| 54 | CF₃ | N | Propargyl | Br | I |
| 55 | CF₃ | N | Propargyl | Br | CF₃ |
| 56 | CF₃ | N | Propargyl | I | CF₃ |
| 57 | H | CH | Allyl | Br | OCHF₂ |
| 58 | H | CH | Allyl | I | OCHF₂ |
| 59 | F | CH | Allyl | Br | OCHF₂ |
| 60 | F | CH | Allyl | I | OCHF₂ |
| 61 | CN | CH | Allyl | Br | OCHF₂ |
| 62 | CN | CH | Allyl | I | OCHF₂ |
| 63 | CF₃ | CH | Allyl | Br | OCHF₂ |
| 64 | CF₃ | CH | Allyl | I | OCHF₂ |
| 65 | F | N | Allyl | Br | OCHF₂ |
| 66 | F | N | Allyl | I | OCHF₂ |
| 67 | CN | N | Allyl | Br | OCHF₂ |
| 68 | CN | N | Allyl | I | OCHF₂ |
| 69 | CF₃ | N | Allyl | Br | OCHF₂ |
| 70 | CF₃ | N | Allyl | I | OCHF₂ |
| 71 | H | CH | Propargyl | Br | OCHF₂ |
| 72 | H | CH | Propargyl | I | OCHF₂ |
| 73 | F | CH | Propargyl | Br | OCHF₂ |
| 74 | F | CH | Propargyl | I | OCHF₂ |
| 75 | CN | CH | Propargyl | Br | OCHF₂ |
| 76 | CN | CH | Propargyl | I | OCHF₂ |
| 77 | CF₃ | CH | Propargyl | Br | OCHF₂ |
| 78 | CF₃ | CH | Propargyl | I | OCHF₂ |
| 79 | F | N | Propargyl | Br | OCHF₂ |
| 80 | F | N | Propargyl | I | OCHF₂ |
| 81 | CN | N | Propargyl | Br | OCHF₂ |
| 82 | CN | N | Propargyl | I | OCHF₂ |
| 83 | CF₃ | N | Propargyl | Br | OCHF₂ |
| 84 | CF₃ | N | Propargyl | I | OCHF₂. |

In a possible embodiment, the amide compound is selected from compounds in Table 2. The compounds in Table 2 have the structure shown in General Formula I, and R₁, X, R₂, R₃, and R₄ are shown in Table 2:

TABLE 2

| Compound | | | | | |
|---|---|---|---|---|---|
| Number | R₁ | X | R₂ | R₃ | R₄ |
| 1 | H | CH | Allyl | Br | Br |
| 2 | H | CH | Allyl | Br | I |
| 3 | H | CH | Allyl | Br | CF₃ |
| 4 | H | CH | Allyl | I | CF₃ |
| 5 | F | CH | Allyl | Br | Br |
| 6 | F | CH | Allyl | Br | I |
| 7 | F | CH | Allyl | Br | CF₃ |
| 8 | F | CH | Allyl | I | CF₃ |
| 9 | CN | CH | Allyl | Br | Br |
| 10 | CN | CH | Allyl | Br | I |
| 11 | CN | CH | Allyl | Br | CF₃ |
| 12 | CN | CH | Allyl | I | CF₃ |
| 13 | CF₃ | CH | Allyl | Br | Br |
| 14 | CF₃ | CH | Allyl | Br | I |
| 15 | CF₃ | CH | Allyl | Br | CF₃ |
| 16 | CF₃ | CH | Allyl | I | CF₃ |
| 17 | F | N | Allyl | Br | Br |
| 18 | F | N | Allyl | Br | I |
| 19 | F | N | Allyl | Br | CF₃ |
| 20 | F | N | Allyl | I | CF₃ |
| 21 | CN | N | Allyl | Br | Br |
| 22 | CN | N | Allyl | Br | I |
| 23 | CN | N | Allyl | Br | CF₃ |
| 24 | CN | N | Allyl | I | CF₃ |
| 25 | CF₃ | N | Allyl | Br | Br |
| 26 | CF₃ | N | Allyl | Br | I |
| 27 | CF₃ | N | Allyl | Br | CF₃ |
| 28 | CF₃ | N | Allyl | I | CF₃ |
| 29 | H | CH | Propargyl | Br | Br |
| 30 | H | CH | Propargyl | Br | I |
| 31 | H | CH | Propargyl | Br | CF₃ |
| 32 | H | CH | Propargyl | I | CF₃ |
| 33 | F | CH | Propargyl | Br | Br |
| 34 | F | CH | Propargyl | Br | I |
| 35 | F | CH | Propargyl | Br | CF₃ |
| 36 | F | CH | Propargyl | I | CF₃ |
| 37 | CN | CH | Propargyl | Br | Br |
| 38 | CN | CH | Propargyl | Br | I |
| 39 | CN | CH | Propargyl | Br | CF₃ |
| 40 | CN | CH | Propargyl | I | CF₃ |
| 41 | CF₃ | CH | Propargyl | Br | Br |
| 42 | CF₃ | CH | Propargyl | Br | I |
| 43 | CF₃ | CH | Propargyl | Br | CF₃ |
| 44 | CF₃ | CH | Propargyl | I | CF₃ |
| 45 | F | N | Propargyl | Br | Br |
| 46 | F | N | Propargyl | Br | I |
| 47 | F | N | Propargyl | Br | CF₃ |
| 48 | F | N | Propargyl | I | CF₃ |
| 49 | CN | N | Propargyl | Br | Br |
| 50 | CN | N | Propargyl | Br | I |
| 51 | CN | N | Propargyl | Br | CF₃ |
| 52 | CN | N | Propargyl | I | CF₃ |
| 53 | CF₃ | N | Propargyl | Br | Br |
| 54 | CF₃ | N | Propargyl | Br | I |
| 55 | CF₃ | N | Propargyl | Br | CF₃ |
| 56 | CF₃ | N | Propargyl | I | CF₃. |

In a possible embodiment, the amide compound is selected from compounds in Table 3. The compounds in Table 3 have the structure shown in General Formula I, and R₁, X, R₂, R₃, and R₄ are shown in Table 3:

TABLE 3

| Compound | | | | | |
|---|---|---|---|---|---|
| Number | R₁ | X | R₂ | R₃ | R₄ |
| 3 | H | CH | Allyl | Br | CF₃ |
| 4 | H | CH | Allyl | I | CF₃ |
| 7 | F | CH | Allyl | Br | CF₃ |
| 8 | F | CH | Allyl | I | CF₃ |
| 11 | CN | CH | Allyl | Br | CF₃ |
| 12 | CN | CH | Allyl | I | CF₃ |
| 19 | F | N | Allyl | Br | CF₃ |
| 20 | F | N | Allyl | I | CF₃ |
| 23 | CN | N | Allyl | Br | CF₃ |
| 24 | CN | N | Allyl | I | CF₃ |
| 27 | CF₃ | N | Allyl | Br | CF₃ |
| 28 | CF₃ | N | Allyl | I | CF₃ |
| 31 | H | CH | Propargyl | Br | CF₃ |
| 32 | H | CH | Propargyl | I | CF₃ |
| 35 | F | CH | Propargyl | Br | CF₃ |
| 36 | F | CH | Propargyl | I | CF₃ |
| 39 | CN | CH | Propargyl | Br | CF₃ |
| 40 | CN | CH | Propargyl | I | CF₃ |
| 47 | F | N | Propargyl | Br | CF₃ |
| 48 | F | N | Propargyl | I | CF₃ |
| 51 | CN | N | Propargyl | Br | CF₃ |
| 52 | CN | N | Propargyl | I | CF₃ |
| 55 | CF₃ | N | Propargyl | Br | CF₃ |
| 56 | CF₃ | N | Propargyl | I | CF₃. |

The present invention further includes an intermediate compound for preparing the above amide compound (namely the compound in General Formula I), and the intermediate compound is shown in General Formula II:

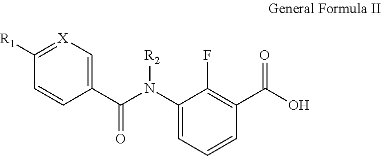

in General Formula II:
R₁ is selected from hydrogen, fluorine, cyano, trifluoromethyl or difluoromethyl;
R₂ is selected from allyl or propargyl; and
X is selected from CH or N.

In a possible embodiment, the intermediate compound is selected from compounds in Table 4. The compounds in Table 4 have the structure shown in General formula II, and R₁, X, and R₂ are shown in Table 4.

TABLE 4

| Compound Number | R₁ | X | R₂ |
|---|---|---|---|
| II.1 | H | CH | Allyl |
| II.2 | F | CH | Allyl |
| II.3 | CN | CH | Allyl |
| II.4 | F | N | Allyl |
| II.5 | CN | N | Allyl |
| II.6 | CF₃ | N | Allyl |
| II.7 | H | CH | Propargyl |
| II.8 | F | CH | Propargyl |
| II.9 | CN | CH | Propargyl |
| II.10 | F | N | Propargyl |
| II.11 | CN | N | Propargyl |
| II.12 | CF₃ | N | Propargyl. |

The present invention further includes an intermediate compound for preparing the above compound in General Formula II, and the intermediate compound is shown in General Formula III:

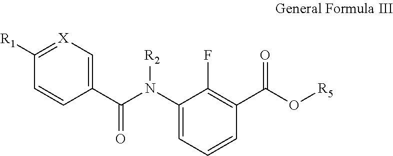

in General Formula III:
R₁ is selected from hydrogen, fluorine, cyano, trifluoromethyl or difluoromethyl;
X is selected from CH or N;
R₂ is selected from allyl or propargyl; and
R₅ is selected from C₁-C₆ alkyl.

In a possible embodiment, the intermediate compound is selected from compounds in Table 5. The compounds in Table 5 have the structure shown in General Formula III, and R₁, X, R₂, and R₅ are shown in Table 5:

TABLE 5

| Compound | | | | |
|---|---|---|---|---|
| Number | R₁ | X | R₂ | R₅ |
| III.1 | H | CH | Allyl | CH₃ |
| III.2 | F | CH | Allyl | CH₃ |
| III.3 | CN | CH | Allyl | CH₃ |
| III.4 | F | N | Allyl | CH₃ |
| III.5 | CN | N | Allyl | CH₃ |
| III.6 | CF₃ | N | Allyl | CH₃ |
| III.7 | H | CH | Propargyl | CH₃ |
| III.8 | F | CH | Propargyl | CH₃ |
| III.9 | CN | CH | Propargyl | CH₃ |
| III.10 | F | N | Propargyl | CH₃ |
| III.11 | CN | N | Propargyl | CH₃ |
| III.12 | CF₃ | N | Propargyl | CH₃ |
| III.13 | H | CH | Allyl | CH₂CH₃ |
| III.14 | F | CH | Allyl | CH₂CH₃ |
| III.15 | CN | CH | Allyl | CH₂CH₃ |
| III.16 | F | N | Allyl | CH₂CH₃ |
| III.17 | CN | N | Allyl | CH₂CH₃ |
| III.18 | CF₃ | N | Allyl | CH₂CH₃ |
| III.19 | H | CH | Propargyl | CH₂CH₃ |
| III.20 | F | CH | Propargyl | CH₂CH₃ |
| III.21 | CN | CH | Propargyl | CH₂CH₃ |
| III.22 | F | N | Propargyl | CH₂CH₃ |
| III.23 | CN | N | Propargyl | CH₂CH₃ |
| III.24 | CF₃ | N | Propargyl | CH₂CH₃. |

The present invention further includes an intermediate compound for preparing the above compound in General Formula III, and the intermediate compound is shown in General Formula IV:

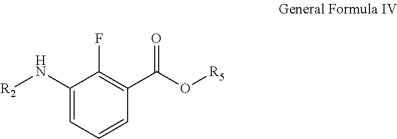

in General Formula IV:
R₂ is selected from allyl or propargyl; and
R₅ is selected from C₁-C₆ alkyl.

In a possible embodiment, the intermediate compound is selected from compounds in Table 6. The compounds in Table 6 have the structure shown in General Formula IV, and R₂ and R₅ are shown in Table 6:

TABLE 6

| Compound | | |
|---|---|---|
| Number | R₂ | R₅ |
| IV.1 | Allyl | CH₃ |
| IV.2 | Allyl | CH₂CH₃ |
| IV.3 | Allyl | CH₂CH₂CH₃ |
| IV.4 | Allyl | CH₂CH₂CH₂CH₃ |
| IV.5 | Allyl | CH₂CH₂CH₂CH₂CH₃ |
| IV.6 | Allyl | CH₂CH₂CH₂CH₂CH₂CH₃ |
| IV.7 | Propargyl | CH₃ |
| IV.8 | Propargyl | CH₂CH₃ |
| IV.9 | Propargyl | CH₂CH₂CH₃ |
| IV.10 | Propargyl | CH₂CH₂CH₂CH₃ |
| IV.11 | Propargyl | CH₂CH₂CH₂CH₂CH₃ |
| IV.12 | Propargyl | CH₂CH₂CH₂CH₂CH₂CH₃. |

The present invention further includes an intermediate compound for preparing the above amide compound (namely the compound in General Formula I), and the intermediate compound is shown in General Formula V:

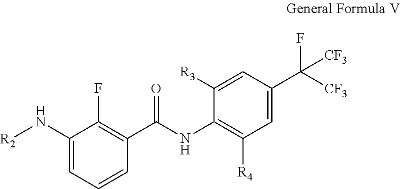

in General Formula V:
R₂ is selected from allyl or propargyl;
R₃ selected from halogen; and
R₄ is selected from halogen, C₁-C₃ haloalkyl, or C₁-C₃ haloalkoxy.

In a possible embodiment, in General Formula V,
R₂ is selected from allyl or propargyl;
R₃ is selected from bromine or iodine; and
R₄ is selected from bromine, iodine, trifluoromethyl or difluoromethoxy.

In a possible embodiment, the intermediate compound is selected from compounds in Table 7. The compounds in Table 7 have the structure shown in General Formula V, and R₂, R₃ and R₄ are shown in Table 7:

TABLE 7

| Compound Number | R₂ | R₃ | R₄ |
|---|---|---|---|
| V.1 | Allyl | Br | Br |
| V.2 | Allyl | Br | I |
| V.3 | Allyl | Br | CF₃ |
| V.4 | Allyl | I | CF₃ |
| V.5 | Allyl | Br | OCHF₂ |
| V.6 | Allyl | I | OCHF₂ |
| V.7 | Propargyl | Br | Br |
| V.8 | Propargyl | Br | I |
| V.9 | Propargyl | Br | CF₃ |
| V.10 | Propargyl | I | CF₃ |
| V.11 | Propargyl | Br | OCHF₂ |
| V.12 | Propargyl | I | OCHF₂ |

In a possible embodiment, the intermediate compound is selected from compounds in Table 8. The compounds in Table 8 have the structure as shown in General formula V, and R₂, R₃ and R₄ are shown in Table 8:

TABLE 8

| Compound Number | R₂ | R₃ | R₄ |
|---|---|---|---|
| V.3 | Allyl | Br | CF₃ |
| V.4 | Allyl | I | CF₃ |
| V.9 | Propargyl | Br | CF₃ |
| V.10 | Propargyl | I | CF₃ |

The present invention further includes an intermediate compound for preparing the above amide compound (namely the compound in General Formula I), and the intermediate compound is shown in General Formula VI:

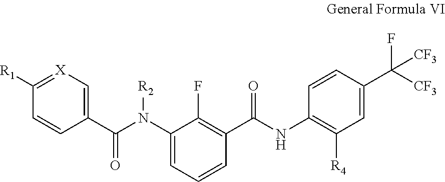

in General Formula VI:
R₁ is selected from hydrogen, fluorine, cyano, trifluoromethyl or difluoromethyl;
R₂ is selected from allyl or propargyl;
R₄ is selected from halogen, C₁-C₃ haloalkyl, or C₁-C₃ haloalkoxy; and
X is selected from CH or N.

In a possible embodiment, in General Formula VI,
R₁ is selected from hydrogen, fluorine, cyano, trifluoromethyl or difluoromethyl;
R₂ is selected from allyl or propargyl;
R₄ is selected from bromine, iodine, trifluoromethyl or difluoromethoxy; and
X is selected from CH or N.

In a possible embodiment, the intermediate compound is selected from compounds in Table 9. The compounds in Table 9 have the structure shown in General Formula VI, and R₁, X, R₂ and R₄ are shown in Table 9:

TABLE 9

| Compound Number | R₁ | X | R₂ | R₄ |
|---|---|---|---|---|
| VI.1 | H | CH | Allyl | CF₃ |
| VI.2 | F | CH | Allyl | CF₃ |
| VI.3 | CN | CH | Allyl | CF₃ |
| VI.4 | F | N | Allyl | CF₃ |
| VI.5 | CN | N | Allyl | CF₃ |
| VI.6 | CF₃ | N | Allyl | CF₃ |
| VI.7 | H | CH | Propargyl | CF₃ |
| VI.8 | F | CH | Propargyl | CF₃ |
| VI.9 | CN | CH | Propargyl | CF₃ |
| VI.10 | F | N | Propargyl | CF₃ |
| VI.11 | CN | N | Propargyl | CF₃ |
| VI.12 | CF₃ | N | Propargyl | CF₃ |
| VI.13 | H | CH | Allyl | OCHF₂ |
| VI.14 | F | CH | Allyl | OCHF₂ |
| VI.15 | CN | CH | Allyl | OCHF₂ |
| VI.16 | F | N | Allyl | OCHF₂ |
| VI.17 | CN | N | Allyl | OCHF₂ |
| VI.18 | CF₃ | N | Allyl | OCHF₂ |
| VI.19 | H | CH | Propargyl | OCHF₂ |
| VI.20 | F | CH | Propargyl | OCHF₂ |
| VI.21 | CN | CH | Propargyl | OCHF₂ |
| VI.22 | F | N | Propargyl | OCHF₂ |
| VI.23 | CN | N | Propargyl | OCHF₂ |
| VI.24 | CF₃ | N | Propargyl | OCHF₂ |

In a possible embodiment, the intermediate compound is selected from compounds in Table 10. The compounds in Table 10 have the structure shown in General Formula VI, and R₁, X, R₂ and R₄ are shown in Table 10:

TABLE 10

| Compound Number | R₁ | X | R₂ | R₄ |
|---|---|---|---|---|
| VI.1 | H | CH | Allyl | CF₃ |
| VI.2 | F | CH | Allyl | CF₃ |
| VI.3 | CN | CH | Allyl | CF₃ |
| VI.4 | F | N | Allyl | CF₃ |
| VI.5 | CN | N | Allyl | CF₃ |
| VI.6 | CF₃ | N | Allyl | CF₃ |
| VI.7 | H | CH | Propargyl | CF₃ |
| VI.8 | F | CH | Propargyl | CF₃ |
| VI.9 | CN | CH | Propargyl | CF₃ |
| VI.10 | F | N | Propargyl | CF₃ |
| VI.11 | CN | N | Propargyl | CF₃ |
| VI.12 | CF₃ | N | Propargyl | CF₃ |

The present invention further includes an intermediate compound for preparing the above compound in General Formula VI, and the intermediate compound is shown in General Formula VII:

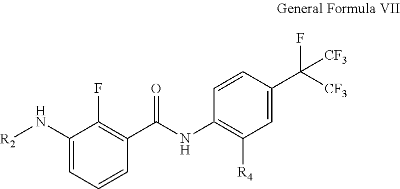

in General Formula VII:
R₂ is selected from allyl or propargyl; and
R₄ is selected from halogen, C₁-C₃ haloalkyl, or C₁-C₃ haloalkoxy.

In a possible embodiment, in General Formula VII,
R₂ is selected from allyl or propargyl; and
R₄ is selected from bromine, iodine, trifluoromethyl or difluoromethoxy.

In a possible embodiment, the intermediate compound is selected from compounds in Table 11. The compounds in Table 11 have the structure shown in General Formula VII, and R₂ and R₄ are shown in Table 11:

TABLE 11

| Compound Number | R₂ | R₄ |
|---|---|---|
| VII.1 | Allyl | CF₃ |
| VII.2 | Allyl | OCHF₂ |
| VII.3 | Propargyl | CF₃ |
| VII.4 | Propargyl | OCHF₂ |

The embodiments of the present invention further provide preparation methods for the above amide compound, including five schemes as follows (each functional group in the formulas is defined as above, unless otherwise specified, LG=Cl, Br or I in the formulas):

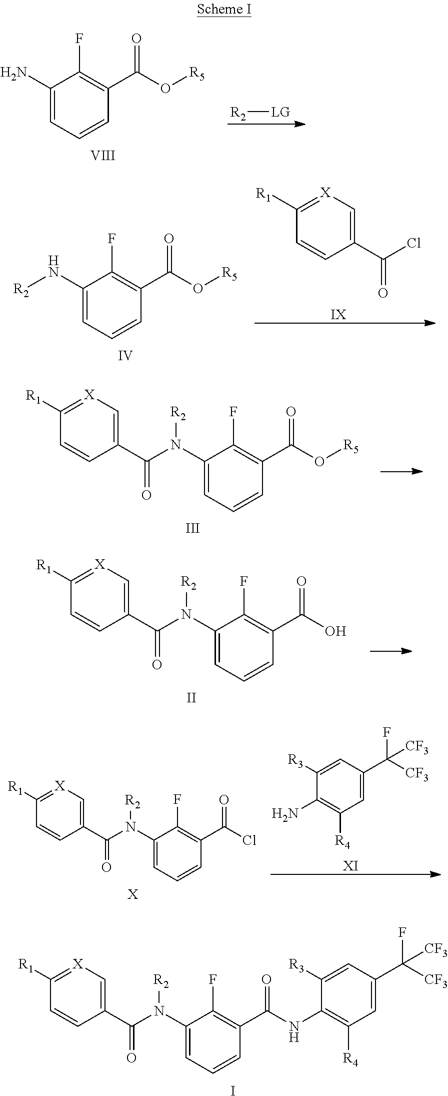

### Step 1: Preparation of Compound of General Formula IV

A compound of General Formula VIII reacts with a compound of General Formula R₂-LG in a suitable solvent at a temperature from −10° C. to the boiling point of the solvent for 0.5-48 h to prepare a compound of General Formula IV, and the reaction is performed in the presence of an alkali.

### Step 2: Preparation of Compound of General Formula III

The compound of General Formula IV reacts with a compound of General Formula IX in a suitable solvent at a temperature from −10° C. to the boiling point of the solvent for 0.5-48 h to prepare a compound of General Formula III, and the reaction is performed in the presence of an alkali or a catalyst.

### Step 3: Preparation of Compound of General Formula II

The compound of General Formula III reacted at a temperature from −10° C. to the boiling point of a solvent for 0.5-48 h in the presence of an alkali to prepare a compound of General Formula II by hydrolyzation: the suitable alkali may be lithium hydroxide, sodium hydroxide, potassium hydroxide, sodium carbonate, potassium carbonate, sodium bicarbonate, potassium bicarbonate, a mixture of lithium bromide and triethylamine, or a mixture of sodium bromide and triethylamine; and the suitable solvent may be any one or a mixed solvent of at least two of water, methanol, ethanol, tetrahydrofuran, and dioxane.

### Step 4: Preparation of Compound of General Formula X

By using a conventional method, the compound of General Formula II can react with thionyl chloride, oxalyl chloride, carbonyl chloride, phosphoryl chloride, phosphorus pentachloride, phosphorus trichloride, triphosgene and the like, to prepare a compound of General Formula X.

### Step 5: Preparation of Compound of General Formula I

The compound of General Formula X reacts with a compound of General Formula XI in a suitable solvent at a temperature from −70° C. to the boiling point of the solvent for 0.5-48 h to prepare the compound of General Formula I, and the reaction is performed in the presence of an alkali or a catalyst.

In Steps 1, 2, and 5, the suitable solvent may be aromatic hydrocarbons such as benzene, toluene, and xylene, ketones such as acetone, methyl ethyl ketone, and methyl isobutyl ketone, halogenated hydrocarbons such as chloroform and dichloromethane, esters such as methyl acetate and ethyl acetate, ethers such as tetrahydrofuran, dioxane, diethyl ether, 1,2-dimethoxyethane, polar solvents such as water, acetonitrile, N,N-dimethylformamide, N-methylpyrrolidone, and dimethyl sulfoxide or mixed solvents of the above solvents; the alkali may be the same or different, including organic alkalis such as trimethylamine, triethylamine, pyridine, DBU, 4-dimethylaminopyridine, N,N-diisopropylmethylamine, and N,N-diisopropylethylamine, alkali metal hydrides such as sodium hydride and potassium hydride, alkali metal hydroxides such as sodium hydroxide and potassium hydroxide, alkaline earth metal hydroxides such as calcium hydroxide, alkali metal carbonates such as sodium carbonate and potassium carbonate, alkali metal bicarbonates such as sodium bicarbonate, and metal alkoxides such as sodium methoxide, sodium ethoxide, potassium ethoxide, potassium tert-butoxide, and sodium tert-butoxide; and the catalyst may be the same or different, including potassium iodide, sodium iodide, potassium fluoride, sodium fluoride, potassium bromide, or sodium bromide or the like.

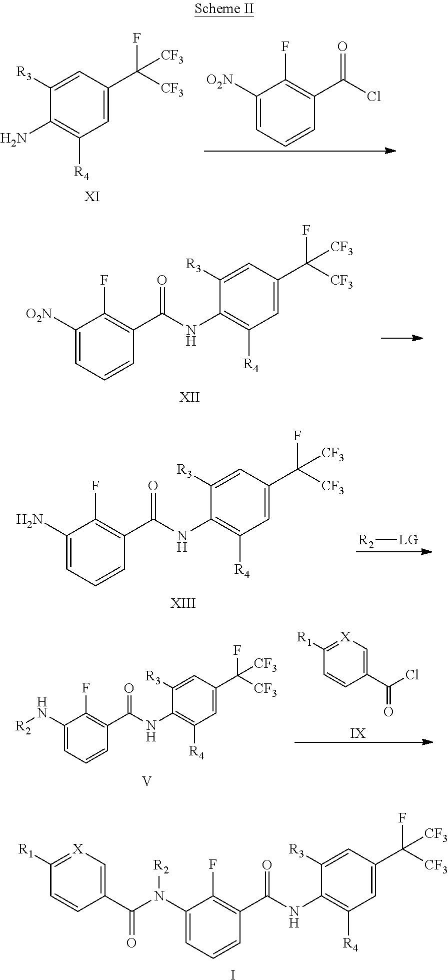

### Step 1: Preparation of Compound of General Formula XII

A compound of General Formula XI reacts with 2-fluoro-3-nitrobenzoyl chloride in a suitable solvent at a temperature from −10° C. to the boiling point of the solvent for 0.5-48 h to prepare a compound of General Formula XII, and the reaction is performed in the presence of an alkali or a catalyst.

### Step 2: Preparation of Compound of General Formula XIII

A compound of General Formula XIII is prepared by a reduction reaction of the compound of General Formula XII.

As the reduction reaction, a method of utilizing a hydrogenation reaction and a method of utilizing a metal compound (such as stannous chloride) or a metal (zinc powder, iron powder and the like) may be listed.

The method of utilizing a hydrogenation reaction may conduct a reaction in an appropriate solvent, in the presence of a catalyst, under normal pressure or pressure, and in a hydrogen atmosphere. As the catalyst in the hydrogenation reaction, it may be a palladium catalyst such as palladium-carbon, a cobalt catalyst, a rhodium catalyst, a platinum catalyst and the like. As the solvent, it may be alcohols such as methanol and ethanol; aromatic hydrocarbons such as benzene and toluene: chained or cyclic ethers such as acetaldehyde and tetrahydrofuran; and esters such as ethyl acetate. Preferably, the pressure of the hydrogenation reaction is 0.1-10 MPa, such as 0.1 MPa, 0.5 MPa, 0.8 MPa, 1 MPa, 1.5 MPa, 2 MPa, 3 MPa, 4 MPa, 5 MPa, 6 MPa, 7 MPa, 8 MPa, 9 MPa, or 10 MPa.

Preferably, the temperature of the hydrogenation reaction is greater than or equal to −20° C. and less than or equal to the boiling point of the reaction solvent, such as −20° C., −10° C., −5° C., 0° C., 5° C., 10° C., 15° C., 20° C., 25° C., 30° C., 35° C., 40° C., 45° C., 50° C., 60° C., 70° C., 75° C., or 80° C., or the reaction is performed at the solvent boiling point, namely in a reflux state.

Preferably, the time of the hydrogenation reaction is 0.5-48 h, such as 0.5 h, 1 h, 3 h, 5 h, 8 h, 10 h, 12 h, 15 h, 18 h, 20 h, 23 h, 25 h, 28 h, 30 h, 33 h, 35 h, 38 h, 40 h, 44 h, or 48 h.

Preferably, the method of utilizing a metal compound or a metal is performed in any one or a mixed solvent of at least two of methanol, ethanol, and ethyl acetate.

Preferably, the metal compound is stannous chloride, and the metal is any one or a combination of at least two of zinc powder and iron powder.

Preferably, the reaction temperature of the method of utilizing a metal compound or a metal is greater than or equal to −10° C. and less than or equal to the boiling point of the reaction solvent, such as −10° C., −5° C., 0° C., 5° C., 10° C., 15° C., 20° C., 25° C., 30° C., 35° C., 40° C., 45° C., 50° C., 60° C., 70° C., 75° C., or 80° C., or the reaction is performed at the solvent boiling point, namely in the reflux state.

Preferably, the reaction time of the method of utilizing a metal compound or a metal is 0.5-48 h, such as 0.5 h, 1 h, 3 h, 5 h, 8 h, 10 h, 12 h, 15 h, 18 h, 20 h, 23 h, 25 h, 28 h, 30 h, 33 h, 35 h, 38 h, 40 h, 44 h, or 48 h.

### Step 3: Preparation of Compound of General Formula V

A compound of General Formula XIII reacts with a compound of General Formula R₂-LG in a suitable solvent at a temperature from −10° C. to the boiling point of the solvent for 0.5-48 h to prepare a compound of General Formula V, and the reaction is performed in the presence of an alkali or a catalyst.

### Step 4: Preparation of Compound of General Formula I

The compound of General Formula V reacts with a compound of General Formula IX in a suitable solvent at a temperature from −10° C. to the boiling point of the solvent for 0.5-48 h to prepare the compound of General Formula I, and the reaction is performed in the presence of an alkali or a catalyst.

In Steps 1, 3, and 4, the suitable solvent may be the same or different, including aromatic hydrocarbons such as benzene, toluene, and xylene, ketones such as acetone, methyl ethyl ketone, and methyl isobutyl ketone, halogenated hydrocarbons such as chloroform and dichloromethane, esters such as methyl acetate and ethyl acetate, ethers such as tetrahydrofuran, dioxane, diethyl ether, 1,2-dimethoxyethane, polar solvents such as water, acetonitrile, N,N-dimethylformamide, N-methylpyrrolidone, and dimethyl sulfoxide or mixed solvents of the above solvents: the alkali may be the same or different, including organic alkalis such as trimethylamine, triethylamine, pyridine, DBU, 4-dimethylaminopyridine, N,N-diisopropylmethylamine, and N,N-diisopropylethylamine, alkali metal hydrides such as sodium hydride and potassium hydride, alkali metal hydroxides such as sodium hydroxide and potassium hydroxide, alkaline earth metal hydroxides such as calcium hydroxide, alkali metal carbonates such as sodium carbonate and potassium carbonate, alkali metal bicarbonates such as sodium bicarbonate, and metal alkoxides such as sodium methoxide, sodium ethoxide, potassium ethoxide, potassium tert-butoxide, and sodium tert-butoxide; and the catalyst may be the same or different, including potassium iodide, sodium iodide, potassium fluoride, sodium fluoride, potassium bromide, or sodium bromide or the like.

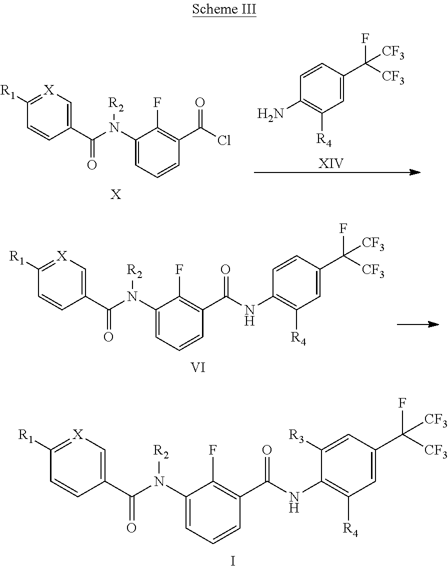

### Step 1: Preparation of Compound of General Formula VI

A compound of General Formula X reacts with a compound of General Formula XIV in a suitable solvent at a temperature from −10° C. to the boiling point of the solvent for 0.5-48 h to prepare a compound of General Formula VI: and an appropriate amount of an alkali or a catalyst may be added to facilitate the reaction. The suitable solvent may be aromatic hydrocarbons such as benzene, toluene, and xylene, ketones such as acetone, methyl ethyl ketone, and methyl isobutyl ketone, halogenated hydrocarbons such as chloroform and dichloromethane, esters such as methyl acetate and ethyl acetate, ethers such as tetrahydrofuran, dioxane, diethyl ether, 1,2-dimethoxyethane, polar solvents such as water, acetonitrile, N,N-dimethylformamide, N-methylpyrrolidone, and dimethyl sulfoxide or mixed solvents of the above solvents; the alkali may be the same or different, including organic alkalis such as trimethylamine, triethylamine, pyridine, DBU, 4-dimethylaminopyridine, N,N-diisopropylmethylamine, and N,N-diisopropylethylamine, alkali metal hydrides such as sodium hydride and potassium hydride, alkali metal hydroxides such as sodium hydroxide and potassium hydroxide, alkaline earth metal hydroxides such as calcium hydroxide, alkali metal carbonates such as sodium carbonate and potassium carbonate, alkali metal bicarbonates such as sodium bicarbonate, and metal alkoxides such as sodium methoxide, sodium ethoxide, potassium ethoxide, potassium tert-butoxide, and sodium tert-butoxide: and the catalyst may be the same or different, including potassium iodide, sodium iodide, potassium fluoride, sodium fluoride, potassium bromide, or sodium bromide or the like.

### Step 2: Preparation of Compound of General Formula I

The compound of General Formula VI reacts with a suitable halogenated reagent in a suitable solvent to prepare the compound of General Formula I.

The reaction usually requires the participation of a suitable alkali, and the suitable alkali may be selected from trimethylamine, triethylamine, pyridine, DBU, 4-dimethylaminopyridine, N,N-diisopropylmethylamine, N,N-diisopropylethylamine, sodium hydride, potassium hydride, sodium hydroxide, potassium hydroxide, calcium hydroxide, sodium carbonate, potassium carbonate, sodium bicarbonate, potassium bicarbonate, sodium methoxide, sodium ethoxide, potassium ethoxide, potassium tert-butoxide or sodium tert-butoxide; and preferably sodium hydride, potassium hydride, sodium hydroxide, or potassium hydroxide.

The suitable halogenated reagent is selected from chlorine gas, liquid bromine, iodine, NBS, NCS, NIS, a mixture of hydrogen peroxide and hydrobromic acid, a mixture of hydrogen peroxide and hydroiodic acid, a mixture of sodium hypochlorite and hydrobromic acid, a mixture of sodium hypochlorite and hydroiodic acid, a mixture of sodium chlorate and hydrobromic acid, or a mixture of sodium chlorate and hydroiodic acid.

The suitable solvent is selected from benzene, toluene, xylene, acetone, methyl ethyl ketone, methyl isobutyl ketone, chloroform, dichloromethane, methyl acetate, ethyl acetate, tetrahydrofuran, dioxane, diethyl ether, 1,2-dimethoxyethane, water, acetonitrile,

N,N-dimethylformamide, N,N-dimethylacetamide, N-methylpyrrolidone, dimethyl sulfoxide or mixed solvents of the above solvents.

The reaction temperature is −10° C. to the boiling point of the solvent selected; and preferably the reaction temperature is 0° C.-100° C., and more preferably 25° C.-80° C.

The reaction time is 0.5-48 h, preferably 1-10 h.

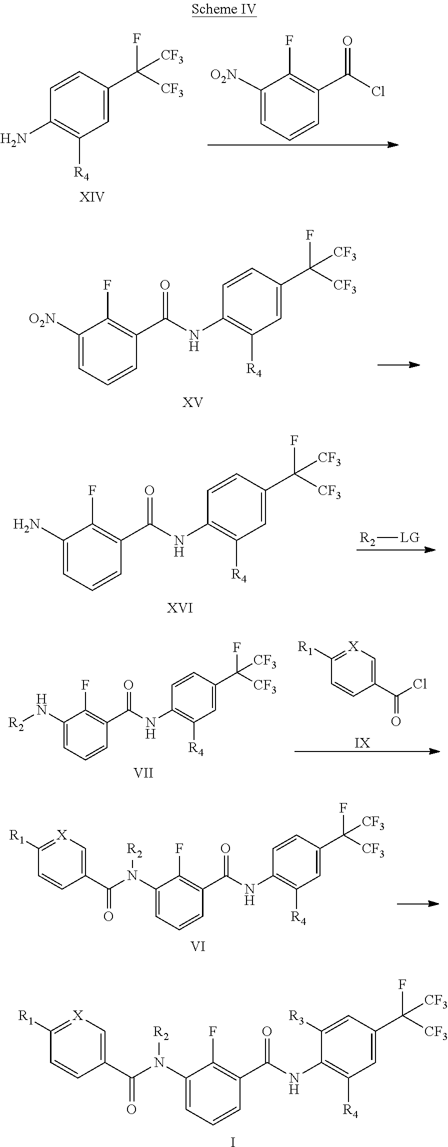

### Step 1: Preparation of Compound of General Formula XV

A compound of General Formula XIV reacts with 2-fluoro-3-nitrobenzoyl chloride in a suitable solvent at a temperature from −10° C. to the boiling point of the solvent for 0.5-48 h to prepare a compound of General Formula XV, and the reaction is performed in the presence of an alkali or a catalyst.

### Step 2: Preparation of Compound of General Formula XVI

A compound of General Formula XVI is prepared by a reduction reaction of the compound of General Formula XV.

As the reduction reaction, a method of utilizing a hydrogenation reaction and a method of utilizing a metal compound (such as stannous chloride) or a metal (zinc powder, iron powder and the like) may be listed.

The method of utilizing a hydrogenation reaction may conduct a reaction in an appropriate solvent, in the presence of a catalyst, under normal pressure or pressure, and in a hydrogen atmosphere. As the catalyst in the hydrogenation reaction, it may be a palladium catalyst such as palladium-carbon, a cobalt catalyst, a rhodium catalyst, a platinum catalyst and the like. As the solvent, it may be alcohols such as methanol and ethanol; aromatic hydrocarbons such as benzene and toluene; chained or cyclic ethers such as acetaldehyde and tetrahydrofuran; and esters such as ethyl acetate. Preferably, the pressure of the hydrogenation reaction is 0.1-10 MPa, such as 0.1 MPa, 0.5 MPa, 0.8 MPa, 1 MPa, 1.5 MPa, 2 MPa, 3 MPa, 4 MPa, 5 MPa, 6 MPa, 7 MPa, 8 MPa, 9 MPa, or 10 MPa.

Preferably, the temperature of the hydrogenation reaction is greater than or equal to −20° C. and less than or equal to the boiling point of the reaction solvent, such as −20° C., −10° C., −5° C., 0° C., 5° C., 10° C., 15° C., 20° C., 25° C., 30° C., 35° C., 40° C., 45° C., 50° C., 60° C., 70° C., 75° C., or 80° C., or the reaction is performed at the solvent boiling point, namely in a reflux state.

Preferably, the time of the hydrogenation reaction is 0.5-48 h, such as 0.5 h, 1 h, 3 h, 5 h, 8 h, 10 h, 12 h, 15 h, 18 h, 20 h, 23 h, 25 h, 28 h, 30 h, 33 h, 35 h, 38 h, 40 h, 44 h, or 48 h.

Preferably, the method of utilizing a metal compound or a metal is performed in any one or a mixed solvent of at least two of methanol, ethanol, and ethyl acetate.

Preferably, the metal compound is stannous chloride, and the metal is any one or a combination of at least two of zinc powder and iron powder.

Preferably, the reaction temperature of the method of utilizing a metal compound or a metal is greater than or equal to −10° C. and less than or equal to the boiling point of the reaction solvent, such as −10° C., −5° C., 0° C., 5° C., 10° C., 15° C., 20° C., 25° C., 30° C., 35° C., 40° C., 45° C., 50° C., 60° C., 70° C., 75° C., or 80° C., or the reaction is performed at the solvent boiling point, namely in the reflux state.

Preferably, the reaction time of the method of utilizing a metal compound or a metal is 0.5-48 h, such as 0.5 h, 1 h, 3 h, 5 h, 8 h, 10 h, 12 h, 15 h, 18 h, 20 h, 23 h, 25 h, 28 h, 30 h, 33 h, 35 h, 38 h, 40 h, 44 h, or 48 h.

### Step 3: Preparation of Compound of General Formula VII

The compound of General Formula XVI reacts with a compound of General Formula R₂-LG in a suitable solvent at a temperature from −10° C. to the boiling point of the solvent for 0.5-48 h to prepare a compound of General Formula VII, and the reaction is performed in the presence of an alkali or a catalyst.

### Step 4: Preparation of Compound of General Formula VI

The compound of General Formula VII reacts with a compound of General Formula IX in a suitable solvent at a temperature from −10° C. to the boiling point of the solvent for 0.5-48 h to prepare a compound of General Formula VI, and the reaction is performed in the presence of an alkali or a catalyst.

### Step 5: Preparation of Compound of General Formula I

This step is the same as Step 2 of Scheme III.

In Steps 1, 3, and 4, the suitable solvent may be the same or different, including aromatic hydrocarbons such as benzene, toluene, and xylene, ketones such as acetone, methyl ethyl ketone, and methyl isobutyl ketone, halogenated hydrocarbons such as chloroform and dichloromethane, esters such as methyl acetate and ethyl acetate, ethers such as tetrahydrofuran, dioxane, diethyl ether, 1,2-dimethoxyethane, polar solvents such as water, acetonitrile, N,N-dimethylformamide, N-methylpyrrolidone, and dimethyl sulfoxide or mixed solvents of the above solvents; the alkali may be the same or different, including organic alkalis such as trimethylamine, triethylamine, pyridine, DBU, 4-dimethylaminopyridine, N,N-diisopropylmethylamine, and N,N-diisopropylethylamine, alkali metal hydrides such as sodium hydride and potassium hydride, alkali metal hydroxides such as sodium hydroxide and potassium hydroxide, alkaline earth metal hydroxides such as calcium hydroxide, alkali metal carbonates such as sodium carbonate and potassium carbonate, alkali metal bicarbonates such as sodium bicarbonate, and metal alkoxides such as sodium methoxide, sodium ethoxide, potassium ethoxide, potassium tert-butoxide, and sodium tert-butoxide; and the catalyst may be the same or different, including potassium iodide, sodium iodide, potassium fluoride, sodium fluoride, potassium bromide, or sodium bromide or the like.

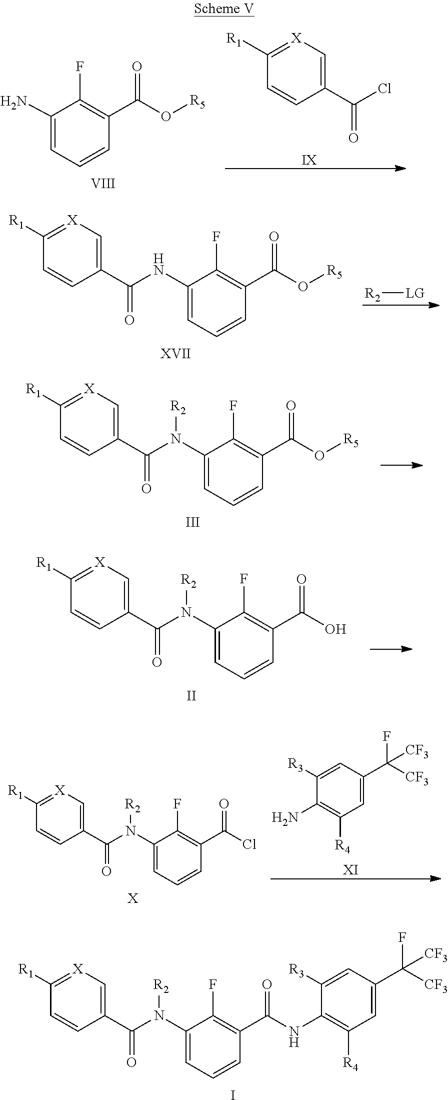

### Step 1: Preparation of Compound of General Formula XVII

A compound of General Formula VIII reacts with a compound of General Formula IX in a suitable solvent at a temperature from −10° C. to the boiling point of the solvent for 0.5-48 h to prepare the compound of General Formula XVII, and the reaction is performed in the presence of an alkali or a catalyst.

### Step 2: Preparation of Compound of General Formula III

The compound of General Formula XVII reacts with a compound of General Formula R₂-LG in a suitable solvent at a temperature from −10° C. to the boiling point of the solvent for 0.5-48 h to prepare a compound of General Formula III, and the reaction is performed in the presence of an alkali or a catalyst.

### Step 3: Preparation of Compound of General Formula II

This step is the same as Step 3 of Scheme I.

### Step 4: Preparation of Compound of General Formula X

This step is the same as Step 4 of Scheme I.

### Step 5: Preparation of Compound of General Formula I

This step is the same as Step 5 of Scheme I.

In Steps 1 and 2, the suitable solvent may be the same or different, including aromatic hydrocarbons such as benzene, toluene, and xylene, ketones such as acetone, methyl ethyl ketone, and methyl isobutyl ketone, halogenated hydrocarbons such as chloroform and dichloromethane, esters such as methyl acetate and ethyl acetate, ethers such as tetrahydrofuran, dioxane, diethyl ether, 1,2-dimethoxyethane, polar solvents such as water, acetonitrile, N,N-dimethylformamide, N-methylpyrrolidone, and dimethyl sulfoxide or mixed solvents of the above solvents; the alkali may be the same or different, including organic alkalis such as trimethylamine, triethylamine, pyridine, DBU, 4-dimethylaminopyridine, N,N-diisopropylmethylamine, and N,N-diisopropylethylamine, alkali metal hydrides such as sodium hydride and potassium hydride, alkali metal hydroxides such as sodium hydroxide and potassium hydroxide, alkaline earth metal hydroxides such as calcium hydroxide, alkali metal carbonates such as sodium carbonate and potassium carbonate, alkali metal bicarbonates such as sodium bicarbonate, and metal alkoxides such as sodium methoxide, sodium ethoxide, potassium ethoxide, potassium tert-butoxide, and sodium tert-butoxide; and the catalyst may be the same or different, including potassium iodide, sodium iodide, potassium fluoride, sodium fluoride, potassium bromide, or sodium bromide or the like.

The compound of General Formula XI and the compound of General Formula XIV may be prepared according to well-known methods, for example, they can be prepared by referring to methods reported in WO20110201687, WO2011093415, WO2005021488, WO2005073165, WO2006137395, JP2007099761, WO2008000438, WO2008074427, WO2008107091, WO2010013567, WO2010018714, WO2010090282, WO2010127926, WO2010127928, JP2011063549, WO2012020483, WO2012020484, WO2012077221, WO2012164698, WO2013050261, WO2014069665, WO2014067838, WO2014161848, WO2014161850, WO2015097091, or WO2015097094; and the compound of General Formula VIII, the compound of General Formula IX, and the compound of General Formula Re-LG are usually available on the market and may also be self-made by using conventional methods.

An embodiment of the present invention further provides a use of the above amide compound in preparation of an insecticide.

In a possible embodiment, the insecticide is used to prevent and control one or more of the following insects:

Beetles (Coleopteran), such as Callosobruchus Chinensis, Sitophilus zeamais, Tribolium Castaneum, Epilachna vigintioctomaculata, Agriotes ogurae fuscicollis, Anomala rufocuprea, Leptinotarsa decemlineata, Diabrotica spp., Monochamus alternatus endai, Lissorhoptrus oryzophilus, and Lyctus bruneus;

Lepidopteran pests, such as Lymantria dispar, Malacosoma neustria, Pieris rapae crucivora, Spodoptera litura, Mamestra brassicae, Chilo suppressalis, Ostrinia nubilalis, Cadra cautella, Adoxophyes honmai, Cydia pomonella, Agrotis segetum, Galleria mellonella, Plutella xylostella, Heliothis virescens, and Phyllocnistis citrella;

Hemipterous pests, such as Nephotettix cincticeps, Nilaparvata lugens, Pseudococcus comstocki, Unaspis yanonensis, Myzus persicae, Aphis pomi, Aphis gossypii, Lipaphis erysimi, Stephanitis nashi, Nezara spp., Trialeurodes vaporariorum, and Pshylla spp.;

Thysanoptera pests, such as Thrips palmi and Franklinella occidentalis;

Orthopteran pests, such as Gryllotalpa Africana and Locusta migratoria;

Blattarian pests, such as Blattella germanica, Periplaneta americana, Reticulitermes speratus, and Coptotermes formosanus;

Dipterous pests, such as Musca domestica, Aedesaegypti, Delia platura, Culex pipiens pallens, Anopheles sinensis, Culex tritaeniorhynchus, and Liriomyza trifolii; and

Agricultural pest mites, such as Tetranychus cinnabarinus, Tetrahychus urticae, Panonychus citri, Aculops pelekassi, and Tarsonemus spp.

In a possible embodiment, the insecticide is used to prevent and control one or more of diamondback moth, Mythimna separata, Spodoptera exigua, Spodoptera litura, Chilo suppressalis, Myzus persicae, thripid, and flea beetle.

An embodiment of the present invention further provides an insecticide formulation, and the insecticide formulation contains the above amide compound as an active component and one or more auxiliary materials.

In a possible embodiment, the insecticide formulation is selected from the following dosage forms: solution, emulsion, wettable powder, granular wettable powder, suspension, powder, foam, paste, tablet, granule, aerosol, natural reagent impregnated with active compounds, synthetic reagent impregnated with active compounds, microcapsule, seed coating agent, preparation equipped with combustion apparatuses (the combustion apparatuses may be a smoke cylinder, a mist cylinder, a can, and a coiler and the like) and ultra-low volume spray (ULV) (cold mist agent and hot mist agent) and the like. These insecticide formulations or animal parasite control agents may be prepared by known methods, such as mixing active component(s) with fillers (such as: a liquid diluent or carrier, a liquefied gas diluent or carrier, and a solid diluent or carrier), and optionally with surfactants (namely an emulsifier and/or a dispersant and/or a foaming agent).

In a possible embodiment, the auxiliary material includes one or more of the following: fillers (such as a liquid diluent or carrier, a liquefied gas diluent or carrier, and a solid diluent or carrier), surfactants (such as an emulsifier and/or a dispersant and/or a foaming agent), adhesives, and coloring agents:
the liquid diluent or carrier may include, for example, aromatic hydrocarbons (xylene, toluene, alkylnaphthalene and the like), chlorinated aromatic hydrocarbons or chlorinated aliphatic hydrocarbons (such as chlorobenzene, vinyl chloride, and dichloromethane), aliphatic hydrocarbons (such as cyclohexane or paraffin (such as a mineral oil fraction)), alcohols (such as butanol, ethylene glycol, and ethers or esters thereof), ketones (such as acetone, methyl ethyl ketone, methyl isobutyl ketone, and cyclohexanone), and strong polar solvents (such as dimethylformamide, and dimethyl sulfoxide), and water. When the water is used as a filler, for example, the organic solvent may be used as a co-solvent;
the liquefied gas diluent or carrier may include those existing in gas form at atmospheric pressure and temperature, such as propane, nitrogen, carbon dioxide, and aerosol propellants such as halogenated hydrocarbons:
the solid diluent may include crushed natural minerals (such as kaolin, clay, talc, chalk, quartz, attapulgite, montmorillonite, or diatomaceous earth) and crushed synthetic minerals (such as finely dispersed silica, alumina, and silicate);
the emulsifier and/or foaming agent may include non-ionic and anionic emulsifiers [such as polyoxyethylene fatty acid esters, polyoxyethylene fatty acid alcohol ethers (such as alkylaryl polyethylene glycol ether), alkyl sulfonate, alkyl sulfate, and aryl sulfonate], and albumin hydrolysis products and the like;
the dispersant may include lignin sulfite waste liquid and methyl cellulose:
the adhesive may include carboxymethyl cellulose, natural or synthetic polymers (such as arabic gum, polyvinyl alcohol, and polyvinyl acetate); and
the coloring agent may include inorganic pigments (such as iron oxide, titanium oxide, and prussian blue), organic dyes such as alizarin dye, azo dye, or metal phthalocyanine dye; and trace elements, such as iron salt, manganese salt, boron salt, copper salt, cobalt salt, molybdenum salt, or zinc salt.

In addition, the amide compound of the present invention may exist as a mixture with a synergist, and the synergist itself does not need to be active. More precisely, it is a compound that enhances the activity of the active compounds.

In a possible embodiment, the amount of the above amide compound contained in the insecticide formulation is 0.1 to 99% by weight, and optionally 0.5 to 90% by weight.

An embodiment of the present invention further provides an insecticide composition, and it includes a mixture of the above amide compound and other active compounds (such as an insecticide, a poison bait agent, a disinfectant, an acaricide, a nematicide, a fungicide, a growth regulator, and a herbicide). The mixture may be provided in a form of active pharmaceutical ingredients, as well as in a form of commercially available preparations or in a use form prepared from its preparations.

An embodiment of the present invention further provides a method for controlling an agricultural or forestal pest, and it includes the following steps: an effective amount of a material is applied to the pest that needs to be controlled or its growth medium, and the material is selected from one or more of the following groups: the above amide compound, the above insecticide formulation, and the above insecticide composition.

An embodiment of the present invention further provides a use of the above amide compound in preparation of an animal parasite control agent. In the field of veterinary medicine, namely in the veterinary science, the amide compound of the present invention may be effectively used to resist various harmful animal parasites, especially in vivo parasites and in vitro parasites.

In a possible embodiment, the animal parasite includes one or more of the following:

Anoplurida, such as Haematopinus spp., Linognathus spp., Pediculus spp., Phtirus spp., and Solenopotes spp.; and specifically, representative examples include Linognathus setosus and Solenopotes capillatus;

Mallopha, such as Linognathus vituli, Linognathus ovillus, Linognathus oviformis, Linognathus pedalis, Linognathus stenopsis, Haematopinus asini macrocephalus, Haematopinus eurysternus, Haematopinus suis, Pediculus humanus capitis, Pediculus humanus corporis, Phylloera vastatrix, and Phthirus pubis, as well as Amblycerina and Ischnocerin, such as Trimenopon spp., Menopon spp., Trinoton spp., Bovicola spp., Werneckiella spp., Lepikentron spp., Damalina spp., Trichodectes spp., and Felicola spp.; and specifically, representative examples include Bovicola bovis, Bovicola ovis, Bovicola limbata, Damalina bovis, Trichodectes canis, Felicola subrostratus, Bovicola caprae, Lepikentron ovis, and Werneckiella equi;

Diptera and its Nematocerina and Brachycerina, such as Aedes spp., Anopheles spp., Culex spp., Simulium spp., Eusimulium spp., Phlebotomus spp., Lutzomyia spp., Culicoides spp., Chrysops spp., Odagmia spp., Wilhelmia spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp., Rhinoestrus spp., and Tipula spp.; and specifically; representative examples include Aedes aegypti, Aedes albopictus, Aedes taeniorhynchus, Anopheles gambiae, Anopheles maculipennis, Calliphora erythrocephala, Chrysozona pluvialis, Culex quinquefasciatus, Culex pipiens, Culex tarsalis, Fannia canicularis, Sarcophaga carnaria, Stomoxys calcitrans, Tipula paludosa, Lucilia cuprina, Lucilia sericata, Simulium reptans, Phlebotomus papatasi, Phlebotomus longipalpis, Odagmia ornata, Wilhelmia equina, Boophthora erythrocephala, Tabanus bromius, Tabanus spodopterus, Tabanus atratus, Tabanus sudeticus, Hybomitra ciurea, Chrysops caecutiens, Chrysops relictus, Haematopota pluvialis, Haematopotaitalica, Musca autumnalis, Musca domestica, Haematobia irritans irritans, Haematobia irritans exigua, Haematobia stimulans, Hydrotaea irritans, Hydrotaea albipuncta, Chrysomya chloropyga, Chrysomya bezziana, Oestrus ovis, Hypoderma bovis, Hypoderma lineatum, Przhevalskiana silenus, Dermatobia hominis, Melophagus ovinus, Lipoptena capreoli, Lipoptena cervi, Hippobosca variegata, Hippobosca equina, Gasterophilus intestinalis, Gasterophilus haemorroidalis, Gasterophilus interrnis, Gasterophilus nasalis, Gasterophilus nigricornis, Gasterophilus pecorum, and Braula coeca;

Siphonapterida, such as Pulex spp., Ctenocephalides spp., Tunga spp., Xenopsylla spp., and Ceratophyllus spp.; and specifically, representative examples include Ctenocephalides canis, Ctenocephalides felis, Pulex irritans, Tunga penetrans, and Xenopsylla cheopis:

Heteropterida, such as Cimex spp., Triatoma spp., Rhodnius spp., and Panstrongylus spp.;

Blattarida, such as Blatta orientalis, American cockroach, Blattella germanica, and Supella spp. (for example, Suppella longipalpa);

Acari (or Acarina), Metastigmata, and Mesostigmata, such as Argas spp., Ornithodorus spp., Otobius spp., Ixodes spp., Amblyomma spp., Rhipicephalus (Boophilus) spp., Dermacentor spp., Haemophysalis spp., Hyalomma spp., Dermanyssus spp., Rhipicephalus spp. (the original genus of heteroxenous mites), Ornithonyssus spp., Pneumonyssus spp., Raillietia spp., Sternostoma spp., Varroa spp., and Acarapis spp.; and specifically, representative examples include Argas persicus, Argas reflexus, Ornithodorus moubata, Otobius megnini, Rhipicephalus (Boophilus) microplus, Rhipicephalus (Boophilus) decoloratus, Rhipicephalus (Boophilus) annulatus, Rhipicephalus (Boophilus) calceratus, Hyalomma anatolicum, Hyalommaaegypticum, Hyalomma marginatum, Hyalomma transiens, Rhipicephalusevertsi, Ixodes ricinus, Ixodes hexagonus, Ixodes canisuga, Ixodes pilosus, Ixodes rubicundus, Ixodes scapularis, Ixodes holocyclus, Haemaphysalis concinna, Haemaphysalis punctata, Haemaphysalis cinnabarina, Haemaphysalis otophila, Haemaphysalis leachi, Haemaphysalis longicorni, Dermacentor marginatus, Dermacentor reticulates, Dermacentor pictus, Dermacentor albipictus, Dermacentor andersoni, Dermacentor variabilis, Hyalomma mauritanicum, Rhipicephalus sanguineus, Rhipicephalus bursa, Rhipicephalus appendiculatus, Rhipicephalus capensis, Rhipicephalus turanicus, Rhipicephalus zambeziensis, Amblyomma americanum, Amblyomma variegatum, Amblyomma maculatum, Amblyomma hebraeum, Amblyomma cajennense, Dermanyssus gallinae, Ornithonyssus bursa, Ornithonyssus sylviarum, and Varroa jacobsconi;

Actinedida (Prostigmata) and Acaridida (Astigmata), such as Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Tyrophagus spp., Pterolichus spp., Psoroptes spp., Chorioptes spp., Otodectes spp., Sarcoptes spp., Notoedres spp., Knemidocoptes spp., Cytodites spp., and Laminosioptes spp.; and especially, Cheyletiella yasguri, Cheyletiella blakei, Demodex canis, Demodex bovis, Demodex ovis, Demodex caprae, Demodex equi, Demodex caballi, Demodex suis, Neotrombicula autumnalis, Neotrombiculadesaleli, Neoschonegastia xerothermobia, Trombicula akamushi, Otodectes cynotis, Notoedres cati, Sarcoptis canis, Sarcoptes bovis, Sarcoptes ovis, Sarcoptes rupicaprae (=S. caprae), Sarcoptes equi, Sarcoptes suis, Psoroptes ovis, Psoroptes cuniculi, Psoroptes equi, Chorioptes bovis, Psoergates ovis, Pneumonyssoidic mange, Pneumonyssoides caninum, and Acarapis woodi:

Nematodes, such as Meloidogyne incognita, Bursaphelenchus xylophilus, Aphelenchoides besseyi, Heterodera glycines, and Pratylenchus spp.; and

Arthropods, worms, and malaria parasites that invade animals. Herein the prevention and control of the arthropods, worms and/or malaria parasites may reduce the mortality rate of domestic animals and improve the animal productivity (meat, milk, hair, skin, egg and honey) and health. In a possible embodiment, the animal parasite control agent is used to prevent and control one or more of cat flea and American dog tick.

In a possible embodiment, the animal includes one or more of the following: agricultural animals, such as cow, sheep, goat, horse, pig, donkey, camel, water buffalo, rabbit, domestic chicken, turkey, duck, goose, farmed fish, and bee; pets known as companion animals, such as dog, cat, cage bird, and ornamental fish; and animals used for experiments, such as hamster, guinea pig, rat, and mouse.

An embodiment of the present invention further provides an animal parasite control agent, and the animal parasite control agent contains the above amide compound which is used as an active component, and further contains one or more of auxiliary materials.

In a possible embodiment, the animal parasite control agent is selected from the following dosage forms: tablet, capsule, potus, drinkable drug, granule, paste, pill, suppository; injection (intramuscular, subcutaneous, intravenous, intraperitoneal and the like), liniment, aerosol, and pressureless spray (such as a pump spray and an atomized spray).

In a possible embodiment, the amount of the above active component contained in the animal parasite control agent is 1 to 80% by weight.

An embodiment of the present invention further provides an animal parasite control composition, and it includes a mixture of the above amide compound and other animal parasite control active compounds (such as an acaricide, an insecticide, a parasiticide, and an antiplasmodial agent). The mixture may be provided in a form of active pharmaceutical ingredients, as well as in a form of commercially available preparations or in a use form prepared from its preparations.

An embodiment of the present invention further provides a method for controlling an animal parasite, and it includes the following steps: an effective amount of a material is applied to the animal parasite that needs to be controlled or its growth medium, and the material is selected from one or more of the following groups: the above amide compound: the above animal parasite control agent: and the above animal parasite control composition. For example: enteral administration by using tablet, capsule, potus, drinkable drug, granule, paste, pill, and suppository: skin based non-enteral administration, such as injection (intramuscular, subcutaneous, intravenous, intraperitoneal and the like), implantation, nasal administration, including bathing or soaking, spraying, pouring, dripping, washing, and powder spraying: and application by using model products containing the active compound, such as collar, ear tag, label, leg brace, net, and marker. The active compound of the present invention has low toxicity and may be safely used in warm blooded animals.

### BENEFICIAL EFFECT

The amide compound of the present invention has unexpected and excellent insecticidal effects, as well as suitable prevention and control effects on toxic pests, and does not have plant toxicity to cultivated crops and plants. In addition, the compound of the present invention can be used to prevent and control various pests, such as harmful sucking insects, chewing insects, and other plant parasitic pests, stored grain pests, and sanitation pests, and can be used for disinfecting and killing them.

### DETAILED DESCRIPTION OF THE INVENTION

In order to make the purposes, technical solutions, and advantages of the examples of the present invention clearer, the technical solutions in the examples of the present invention are clearly and completely described below. Apparently, the examples described are a part of the examples of the present invention, not all of the examples. Based on the examples in the present invention, all other examples obtained by those of ordinary skill in the art without creative labor shall fall within the scope of protection of the present invention.

In addition, in order to describe the present invention better, numerous specific details are provided in the following specific implementation modes. It should be understood by those skilled in the art that, without certain specific details, the present invention may also be implemented. In some examples, raw materials, elements, methods, means and the like familiar to those skilled in the art are not described in detail, as to highlight the main idea of the present invention.

Unless otherwise explicitly stated, throughout the entire description and claims, the term “include(s)/including” or its variations such as “contain(s)/containing” or “comprise(s)/comprising” may be understood to include the stated elements or constituent parts, without excluding other elements or constituent parts.

Unless otherwise specified, all raw materials used are commercially available.

In the present invention, the terms used have the following meanings:
Halogen: referring to fluorine, chlorine, bromine, or iodine.
Haloalkyl: straight or branched alkyls on which hydrogen atoms may be partially or completely replaced by halogen(s), such as difluoromethyl (CHF₂), and trifluoromethyl (CF₃).
Haloalkoxy: hydrogen atoms on alkoxy may be partially or completely replaced by halogen(s), such as difluoromethoxy (OCHF₂), and trifluoromethoxy (OCF₃).
Allyl: —CH₂—CH═CH₂.
Propargyl: —CH₂—C═CH.
Insecticide: a substance that has insecticidal effects on pests.
Animal parasite control agent: referring to an active compound that may effectively reduce the incidence rate of various parasites in animals infected by the parasites. Prevention and control means that the active compound may effectively kill the parasites, and inhibit their growth or reproduction.

### SYNTHESIS EXAMPLE

According to the synthesis routes described above, different raw material compounds may be used to prepare and obtain the compounds shown in General Formulas I-VII of the present invention, and they are further described specifically as follows:

### Example 1: Preparation of IntermediateCcompound II.7

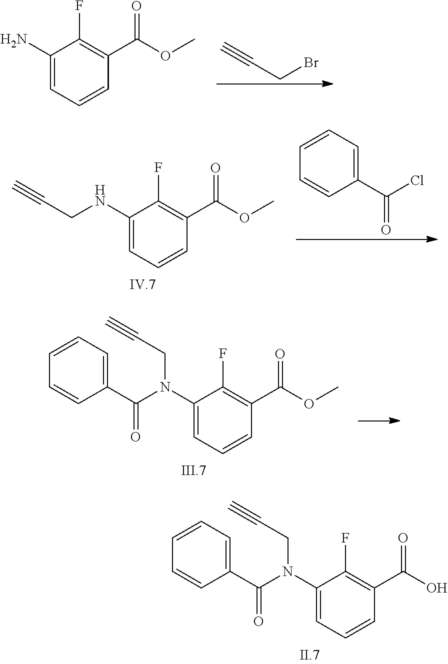

### (1) Preparation of methyl 2-fluoro-3-(N-(prop-2-yn-1-yl) benzoylamino)benzoate (III.7)

0.50 g (2.96 mmol) of methyl 2-fluoro-3-aminobenzoate, 0.70 g (5.94 mmol) of propargyl bromide, and 1.15 g (8.90 mmol) of N,N-diisopropylethylamine were added to 10 mL of toluene, and the temperature was raised for a reflux reaction. The reaction process was monitored by TLC, and after raw materials were reacted completely, 0.58 g (4.14 mmol) of benzoyl chloride was added. After TLC monitored the completion of the reaction, water and ethyl acetate were added for extraction. The organic phase was depressurized and desolventized, and the residue was purified by column chromatography to obtain 0.72 g of a white solid, namely methyl 2-fluoro-3-(N-(prop-2-yn-1-yl) benzoylamino)benzoate (III.7). Nuclear magnetism and mass spectrometry data of the intermediate III.7 are as follows:

¹H NMR (600 MHZ, Chloroform-d) δ 7.82 (t, 1H), 7.42-7.13 (m, 6H), 7.07 (t, 1H), 5.05 (s, 1H), 4.28 (s, 1H), 3.91 (s, 3H), 2.23 (t, 1H). LC-MS (m/z, ESI): 312.10 (M+H)⁺.

### (2) Preparation of 2-fluoro-3-(N-(prop-2-yn-1-yl) benzoylamino) benzoic acid (II.7)

0.58 g (1.86 mmol) of methyl 2-fluoro-3-(N-(prop-2-yn-1-yl) benzoylamino)benzoate, 1.62 g (18.65 mmol) of lithium bromide, 0.94 g (9.29 mmol) of triethylamine, and 0.17 g (9.44 mmol) of water were added to 10 mL of acetonitrile, and the temperature was raised to 50° C. for a reaction. After TLC monitored the completion of the reaction, the pH value was adjusted to about 2-3 with a dilute hydrochloric acid, and water and ethyl acetate were added for extraction. The organic phase was depressurized and desolventized to obtain 0.52 g of a white solid, namely the intermediate II.7. Nuclear magnetism and mass spectrometry data of the intermediate II.7 are as follows:

¹H NMR (600 MHZ, DMSO-d₆) δ 7.79-7.64 (m, 2H), 7.42-7.17 (m, 6H), 4.61 (s, 2H), 3.22 (s, 1H). LC-MS (m/z, ESI): 296.11 (M−H)⁻.

### Example 2: Preparation of Intermediate Compound V.3

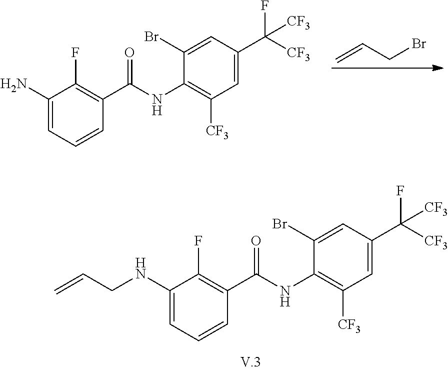

1.00 g (1.84 mmol) of 3-amino-N-(2-bromo-4-(perfluoroprop-2-yl)-6-(trifluoromethyl)phenyl)-2-fluorobenzamide (prepared by referring to a method reported in WO2011093415 or WO2010018714) and 0.30 g (2.00 mmol) of sodium iodide were added to 10 mL of dimethylformamide (DMF), 0.22 g (1.83 mmol) of allyl bromide was dropwise added under stirring, and a reaction was performed at room temperature. After TLC monitored the completion of the reaction, water and ethyl acetate were added for extraction. The organic phase was depressurized and desolventized, and the residue was purified by column chromatography to obtain 0.34 g of a white solid, namely the intermediate compound V.3. Nuclear magnetism and mass spectrometry data of the intermediate compound V.3 are as follows:

¹H NMR (600 MHz, Chloroform-d) δ 8.26 (d, 1H), 8.14 (d, 1H), 7.91 (d, 1H), 7.42-7.36 (m, 1H), 7.14 (t, 1H), 6.95-6.87 (m, 1H), 6.03-5.91 (m, 1H), 5.34 (dd, 1H), 5.24 (dd, 1H), 4.26 (s, 1H), 3.87 (d, 2H). LC-MS (m/z, ESI): 585.08 (M+H)⁺.

### Example 3: Preparation of Intermediate Compound V.4

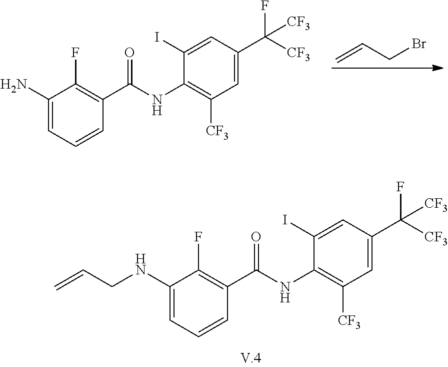

2.00 g (3.38 mmol) of 3-amino-2-fluoro-N-(2-iodo-4-(perfluoroprop-2-yl)-6-(trifluoromethyl)phenyl)benzamide (prepared by referring to a method reported in WO2011093415 or WO2010018714) and 0.30 g (3.81 mmol) of sodium iodide were added to 20 mL of DMF, 0.41 g (3.42 mmol) of allyl bromide was dropwise added under stirring, and a reaction was performed at a room temperature. After TLC monitored the completion of the reaction, water and ethyl acetate were added for extraction. The organic phase was depressurized and desolventized, and the residue was purified by column chromatography to obtain 0.60 g of a white solid, namely the intermediate compound V.4. Nuclear magnetism and mass spectrometry data of the intermediate compound V.4 are as follows:

¹H NMR (600 MHZ, Chloroform-d) δ 8.35 (d, 1H), 8.30 (d, 1H), 7.93 (d, 1H), 7.40 (td, 1H), 7.15 (t, 1H), 6.92 (td, 1H), 6.02-5.92 (m, 1H), 5.34 (dd, 1H), 5.24 (dd, 1H), 4.26 (s, 1H), 3.88 (d, 2H). LC-MS (m/z, ESI): 633.07 (M+H)⁺.

### Example 4: Preparation of Intermediate Compound V.9

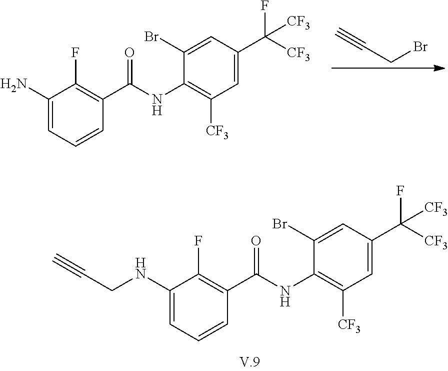

1.00 (1.84 mmol) of 3-amino-N-(2-bromo-4-(perfluoroprop-2-yl)-6-(trifluoromethyl)phenyl)-2-fluorobenzamide and 0.31 g (2.07 mmol) of sodium iodide were added to 10 mL of DMF, 0.22 g (1.87 mmol) of propargyl bromide was dropwise added under stirring, and the temperature was raised to 40° C. for a reaction. After TLC monitored the completion of the reaction, water and ethyl acetate were added for extraction. The organic phase was depressurized and desolventized, and the residue was purified by column chromatography to obtain 0.26 g of a white solid, namely the intermediate compound V.9. Nuclear magnetism and mass spectrometry data of the intermediate compound V.9 are as follows:

¹H NMR (600 MHZ, Chloroform-d) δ 8.24 (d, 1H), 8.14 (d, 1H), 7.91 (d, 1H), 7.51-7.45 (m, 1H), 7.21 (t, 1H), 7.05 (td, 1H), 4.41-4.34 (m, 1H), 4.05 (dd, 2H), 2.28 (t, 1H). LC-MS (m/z, ESI): 583.06 (M+H)⁺.

### Example 5: Preparation of Intermediate Compound V.10

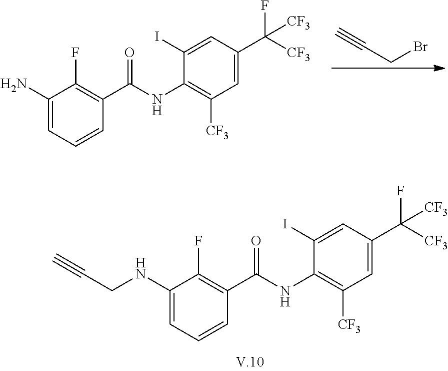

1.00 g (1.69 mmol) of 3-amino-2-fluoro-N-(2-iodo-4-(perfluoroprop-2-yl)-6-(trifluoromethyl)phenyl)benzamide and 0.30 g (2.00 mmol) of sodium iodide were added to 10 mL of DMF, 0.20 g (1.70 mmol) of propargyl bromide was dropwise added under stirring, and the temperature was raised to 40° C. for a reaction. After TLC monitored the completion of the reaction, water and ethyl acetate were added for extraction. The organic phase was depressurized and desolventized, and the residue was purified by column chromatography to obtain 0.31 g of a white solid, namely the intermediate compound V.10. Nuclear magnetism and mass spectrometry data of the intermediate compound V.10 are as follows:

¹H NMR (600 MHz, Chloroform-d) δ 8.35 (d, 1H), 8.28 (d, 1H), 7.96-7.92 (m, 1H), 7.49 (td, 1H), 7.22 (t, 1H), 7.05 (td, 1H), 4.38 (s, 1H), 4.08-4.03 (m, 2H), 2.28 (t, 1H). LC-MS (m/z, ESI): 631.05 (M+H)⁺.

### Example 6: Preparation of Compound 7

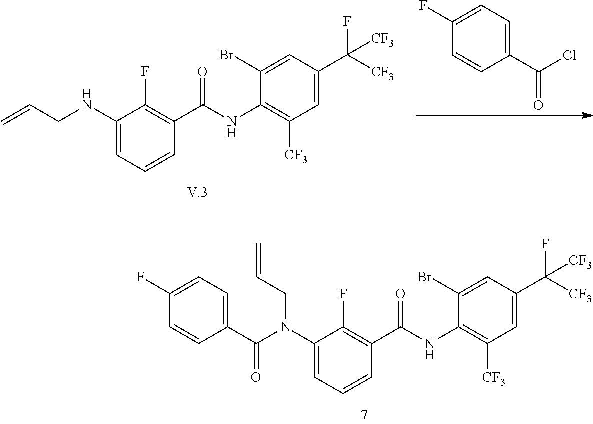 [SMILES: C=CCNc1cccc(C(=O)Nc2c(Br)cc(C(F)(C(F)(F)F)C(F)(F)F)cc2C(F)(F)F)c1F ; O=C(Cl)c1ccc(F)cc1 ; C=CCN(C(=O)c1ccc(F)cc1)c1cccc(C(=O)Nc2c(Br)cc(C(F)(C(F)(F)F)C(F)(F)F)cc2C(F)(F)F)c1F]

0.15 g (0.26 mmol) of an intermediate V.3, 0.04 g (0.27 mmol) of sodium iodide and 0.04 g (0.25 mmol) of para-fluorobenzoyl chloride were added to 10 mL of toluene, and the mixture was heated for reflux. After TLC monitored the completion of the reaction, the reaction product was depressurized and desolventized, and the obtained residue was purified by column chromatography to obtain 0.13 g of a white solid, namely the compound 7. Nuclear magnetism and mass spectrometry data of the compound 7 are as follows:

¹H NMR (600 MHZ, Chloroform-d) δ 8.12 (d, 1H), 8.07-7.95 (m, 2H), 7.89 (d, 1H), 7.51-7.44 (m, 1H), 7.36 (s, 2H), 7.32-7.25 (m, 1H), 6.90 (s, 2H), 6.06-5.92 (m, 1H), 5.26-5.15 (m, 2H), 4.50 (d, 2H). LC-MS (m/z, ESI): 707.04 (M+H)⁺.

### Example 7: Preparation of Compound 8

0.15 g (0.24 mmol) of an intermediate V.4, 0.04 g (0.27 mmol) of sodium iodide, 0.04 g (0.25 mmol) of para-fluorobenzoyl chloride were added to 10 mL of toluene, and the mixture was heated for reflux. After TLC monitored the completion of the reaction, the reaction product was depressurized and desolventized, and the obtained residue was purified by column chromatography to obtain 0.11 g of a white solid, namely the compound 8. Nuclear magnetism and mass spectrometry data of the compound 8 are as follows:

¹H NMR (600 MHZ, Chloroform-d) δ 8.33 (d, 1H), 8.11-7.98 (m, 2H), 7.94-7.89 (m, 1H), 7.48 (dt, 1H), 7.36 (s, 2H), 7.29 (t, 1H), 6.90 (s, 2H), 6.07-5.90 (m, 1H), 5.26-5.15 (m, 2H), 4.52 (d, 2H). LC-MS (m/z, ESI): 755.07 (M+H)⁺.

### Example 8: Preparation of Compound 19

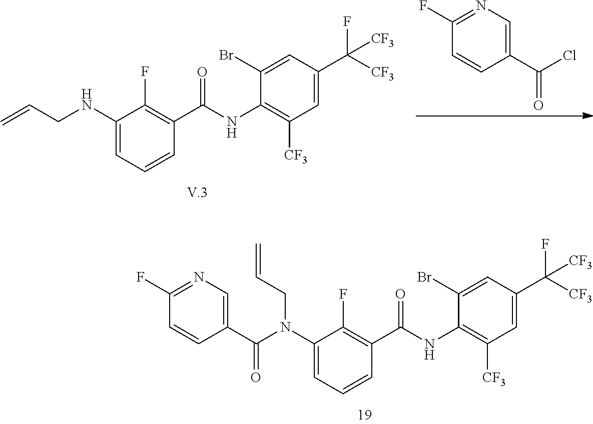 [SMILES: C=CCNc1cccc(C(=O)Nc2c(Br)cc(C(F)(C(F)(F)F)C(F)(F)F)cc2C(F)(F)F)c1F ; O=C(Cl)c1ccc(F)nc1 ; C=CCN(C(=O)c1ccc(F)nc1)c1cccc(C(=O)Nc2c(Br)cc(C(F)(C(F)(F)F)C(F)(F)F)cc2C(F)(F)F)c1F]

0.15 g (0.26 mmol) of an intermediate V.3, 0.06 g (0.40 mmol) of sodium iodide, and 0.06 g (0.38 mmol) of 6-fluoronicotinoyl chloride were added to 10 mL of toluene, and the mixture was heated for reflux. After TLC monitored the completion of the reaction, the reaction product was depressurized and desolventized, and the obtained residue was purified by column chromatography to obtain 0.14 g of a yellow oily substance, namely the compound 19. Nuclear magnetism and mass spectrometry data of the compound 19 are as follows:

¹H NMR (600 MHZ, Chloroform-d) δ 8.18 (s, 1H), 8.13 (d, 1H), 8.08-8.02 (m, 1H), 8.017.79 (m, 3H), 7.54-7.48 (m, 1H), 7.34 (t, 1H), 6.83 (s, 1H), 6.05-5.91 (m, 1H), 5.27-5.18 (m, 2H), 4.53 (d, 2H). LC-MS (m/z, ESI): 708.05 (M+H)⁺.

### Example 9: Preparation of Compound 20

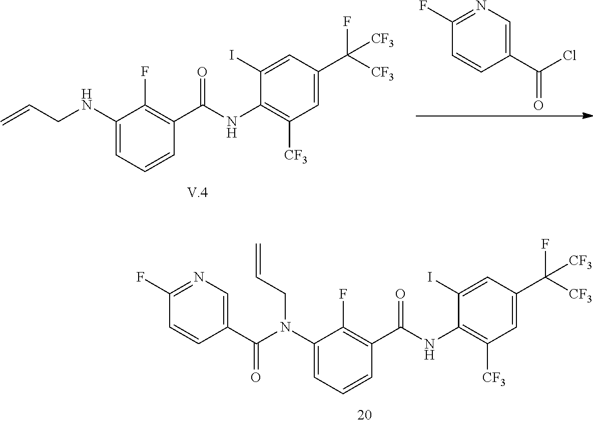 [SMILES: C=CCNc1cccc(C(=O)Nc2c(I)cc(C(F)(C(F)(F)F)C(F)(F)F)cc2C(F)(F)F)c1F ; O=C(Cl)c1ccc(F)nc1 ; C=CCN(C(=O)c1ccc(F)nc1)c1cccc(C(=O)Nc2c(I)cc(C(F)(C(F)(F)F)C(F)(F)F)cc2C(F)(F)F)c1F]

0.15 g (0.24 mmol) of an intermediate V.4, 0.05 g (0.33 mmol) of sodium iodide, and 0.05 g (0.31 mmol) of 6-fluoronicotinoyl chloride were added to 10 mL of toluene, and the mixture was heated for reflux. After TLC monitored the completion of the reaction, the reaction product was depressurized and desolventized, and the obtained residue was purified by column chromatography to obtain 0.09 g of a white solid, namely the compound 20. Nuclear magnetism and mass spectrometry data of the compound 20 are as follows:

¹H NMR (600 MHz, Chloroform-d) δ 8.35-8.31 (m, 1H), 8.19 (s, 1H), 8.09-8.03 (m, 1H), 8.02-7.79 (m, 3H), 7.52 (dt, 1H), 7.35 (t, 1H), 6.83 (s, 1H), 6.05-5.93 (m, 1H), 5.28-5.19 (m, 2H), 4.53 (d, 2H). LC-MS (m/z, ESI): 756.05 (M+H)⁺.

### Example 10: Preparation of Compound 31

### Method I:

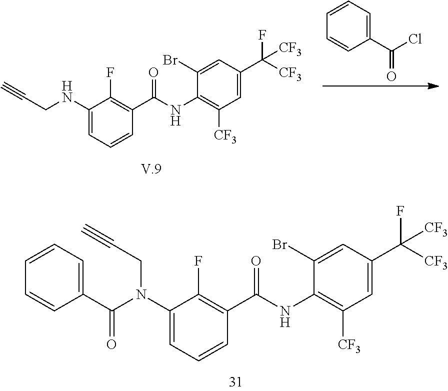 [SMILES: C#CCNc1cccc(C(=O)Nc2c(Br)cc(C(F)(C(F)(F)F)C(F)(F)F)cc2C(F)(F)F)c1F ; O=C(Cl)c1ccccc1 ; C#CCN(C(=O)c1ccccc1)c1cccc(C(=O)Nc2c(Br)cc(C(F)(C(F)(F)F)C(F)(F)F)cc2C(F)(F)F)c1F]

0.15 g (0.26 mmol) of an intermediate V.9, 0.04 g (0.27 mmol) of sodium iodide, and 0.04 g (0.29 mmol) of benzoyl chloride were added to 10 mL of toluene, and the mixture was heated for reflux. After TLC monitored the completion of the reaction, the reaction product was depressurized and desolventized, and the obtained residue was purified by column chromatography to obtain 0.09 g of a yellow oily substance, namely the compound 31. Nuclear magnetism and mass spectrometry data of the compound 31 are as follows:

¹H NMR (600 MHZ, Chloroform-d) δ 8.13 (d, 1H), 8.09-7.95 (m, 2H), 7.92-7.88 (m, 1H), 7.63-7.53 (br, 1H), 7.43-7.14 (m, 6H), 4.93 (s, 1H), 4.51 (s, 1H), 2.30 (t, 1H). LC-MS (m/z, ESI): 687.07 (M+H)⁺.

### Method II:

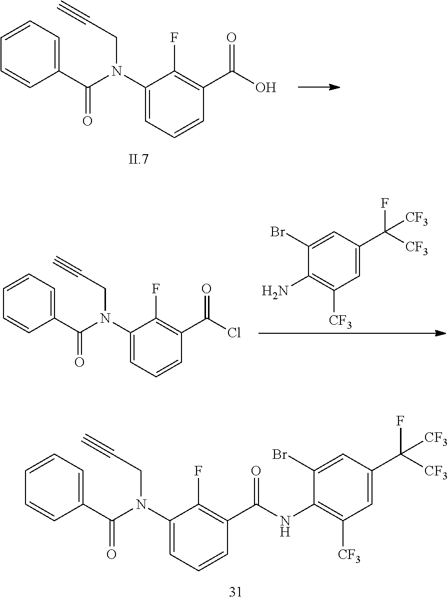 [SMILES: C#CCN(C(=O)c1ccccc1)c1cccc(C(=O)O)c1F ; C#CCN(C(=O)c1ccccc1)c1cccc(C(=O)Cl)c1F ; Nc1c(Br)cc(C(F)(C(F)(F)F)C(F)(F)F)cc1C(F)(F)F ; C#CCN(C(=O)c1ccccc1)c1cccc(C(=O)Nc2c(Br)cc(C(F)(C(F)(F)F)C(F)(F)F)cc2C(F)(F)F)c1F]

0.22 g (0.68 mmol) of an intermediate II.7 and 0.44 g (3.70 mmol) of sulfoxide chloride were added to 10 mL of toluene, and the mixture was heated for reflux for 4 h; and the reaction product was depressurized and desolventized to obtain an intermediate 2-fluoro-3-(N-(prop-2-yn-1-yl)benzoylamino)benzoyl chloride, which was sealed for standby. The intermediate 2-fluoro-3-(N-(prop-2-yn-1-yl)benzoylamino)benzoyl chloride prepared above, 0.08 g (0.78 mmol) of sodium bromide, and 0.30 g (0.74 mmol) of 2-bromo-4-(perfluoroprop-2-yl)-6-(trifluoromethyl)aniline (prepared by referring to a method reported in WO2011093415 or WO2010018714) were added to 10 mL of acetonitrile, and the mixture was heated for reflux. After TLC monitored the completion of the reaction, the reaction product was depressurized and desolventized, and the obtained residue was purified by column chromatography to obtain 0.23 g of a white solid, namely the compound 31. Nuclear magnetism and mass spectrometry data of the compound 31 are as follows:

¹H NMR (600 MHZ, Chloroform-d) δ 8.13 (d, 1H), 8.09-7.95 (m, 2H), 7.92-7.88 (m, 1H), 7.63-7.53 (br, 1H), 7.43-7.14 (m, 6H), 4.93 (s, 1H), 4.51 (s, 1H), 2.30 (t, 1H). LC-MS (m/z, ESI): 687.07 (M+H)⁺.

### Method III:

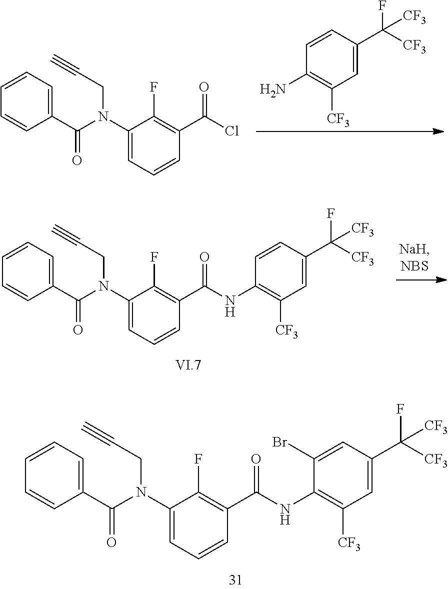 [SMILES: C#CCN(C(=O)c1ccccc1)c1cccc(C(=O)Cl)c1F ; Nc1ccc(C(F)(C(F)(F)F)C(F)(F)F)cc1C(F)(F)F ; C#CCN(C(=O)c1ccccc1)c1cccc(C(=O)Nc2ccc(C(F)(C(F)(F)F)C(F)(F)F)cc2C(F)(F)F)c1F ; C#CCN(C(=O)c1ccccc1)c1cccc(C(=O)Nc2c(Br)cc(C(F)(C(F)(F)F)C(F)(F)F)cc2C(F)(F)F)c1F]

### (1) Preparation of Intermediate Compound VI.7

0.29 g (0.68 mmol) of an intermediate 2-fluoro-3-(N-(prop-2-yn-1-yl) benzoylamino)benzoyl chloride, 0.09 g (0.87 mmol) of sodium bromide, and 0.30 g (0.91 mmol) of 4-(perfluoroprop-2-yl)-2-(trifluoromethyl)aniline (prepared by referring to a method reported in WO2011093415 or WO2010018714) were added to 10 mL of acetonitrile, and the mixture was heated for reflux. After TLC monitored the completion of the reaction, water and ethyl acetate were added for extraction. The organic phase was depressurized and desolventized, and the residue was purified by column chromatography to obtain 0.39 g of a white solid, namely the intermediate compound VI.7.

### (2) Preparation of Compound 31

0.30 g (0.49 mmol) of an intermediate VI.7, 0.02 g (0.50 mmol) of sodium hydride (60% mass fraction), and 0.10 g (0.56 mmol) of N-bromosuccinimide were added to 10 mL of N,N-dimethylformamide, and the temperature was raised to 40° C. for a reaction. After TLC monitored the completion of the reaction, water and ethyl acetate were added for extraction. The organic phase was depressurized and desolventized, and the residue was purified by column chromatography to obtain 0.06 g of a white solid, namely the compound 31. Nuclear magnetism and mass spectrometry data of the compound 31 are as follows:

¹H NMR (600 MHz, Chloroform-d) δ 8.13 (d, 1H), 8.09-7.95 (m, 2H), 7.92-7.88 (m, 1H), 7.63-7.53 (br, 1H), 7.43-7.14 (m, 6H), 4.93 (s, 1H), 4.51 (s, 1H), 2.30 (t, 1H). LC-MS (m/z, ESI): 687.07 (M+H)⁺.

### Method IV:

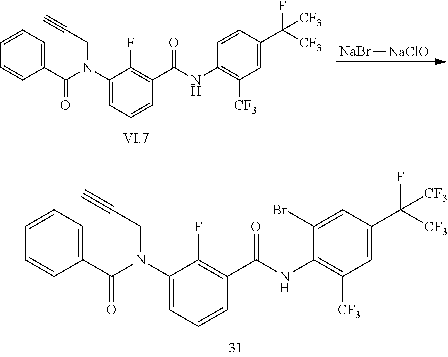

0.50 g (0.82 mmol) of an intermediate VI.7, 0.12 g (1.17 mmol) of sodium bromide, 0.02 g (0.50 mmol) of sodium hydroxide, and 0.21 g of water were added to 10 mL of dichloromethane, and the temperature was raised to 40° C. for a reaction; and 0.64 g (1.21 mmol) of an aqueous solution of sodium hypochlorite (14% mass fraction) was dropwise added to the reaction solution, and a reaction was continuously performed at 40° C. After TLC monitored the completion of the reaction, water and ethyl acetate were added for extraction. The organic phase was depressurized and desolventized, and the residue was purified by column chromatography to obtain 0.23 g of a white solid, namely the compound 31. Nuclear magnetism and mass spectrometry data of the compound 31 are as follows:

¹H NMR (600 MHz, Chloroform-d) δ 8.13 (d, 1H), 8.09-7.95 (m, 2H), 7.92-7.88 (m, 1H), 7.63-7.53 (br, 1H), 7.43-7.14 (m, 6H), 4.93 (s, 1H), 4.51 (s, 1H), 2.30 (t, 1H). LC-MS (m/z, ESI): 687.07 (M+H)⁺.

### Example 11: Preparation of Compound 36

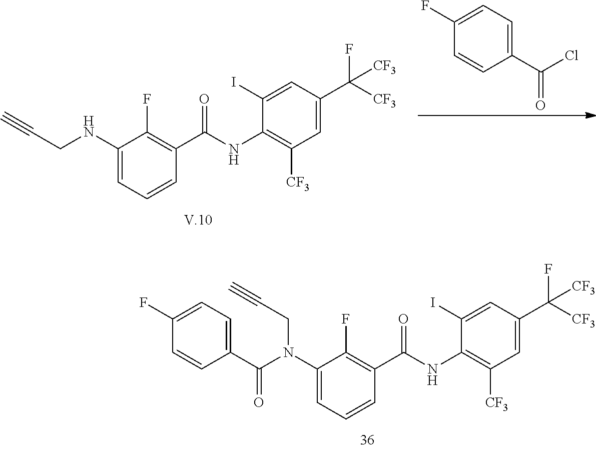 [SMILES: C#CCNc1cccc(C(=O)Nc2c(I)cc(C(F)(C(F)(F)F)C(F)(F)F)cc2C(F)(F)F)c1F ; O=C(Cl)c1ccc(F)cc1 ; C#CCN(C(=O)c1ccc(F)cc1)c1cccc(C(=O)Nc2c(I)cc(C(F)(C(F)(F)F)C(F)(F)F)cc2C(F)(F)F)c1F]

0.15 g (0.24 mmol) of an intermediate V.10, 0.04 g (0.27 mmol) of sodium iodide, and 0.04 g (0.25 mmol) of para-fluorobenzoyl chloride were added to 10 mL of toluene, and the mixture was heated for reflux. After TLC monitored the completion of the reaction, the reaction product was depressurized and desolventized, and the obtained residue was purified by column chromatography to obtain 0.13 g of a yellow oily substance, namely the compound 36. Nuclear magnetism and mass spectrometry data of the compound 36 are as follows:

¹H NMR (600 MHZ, Chloroform-d) δ 8.34 (d, 1H), 8.19-8.01 (m, 2H), 7.98-7.89 (m, 1H), 7.60 (t, 1H), 7.47-7.36 (br s, 2H), 7.33 (t, 1H), 7.03-6.83 (br s, 2H), 4.90 (s, 1H), 4.53 (s, 1H), 2.31 (t, 1H). LC-MS (m/z, ESI): 753.04 (M+H)⁺.

### Example 12: Preparation of Compound 47

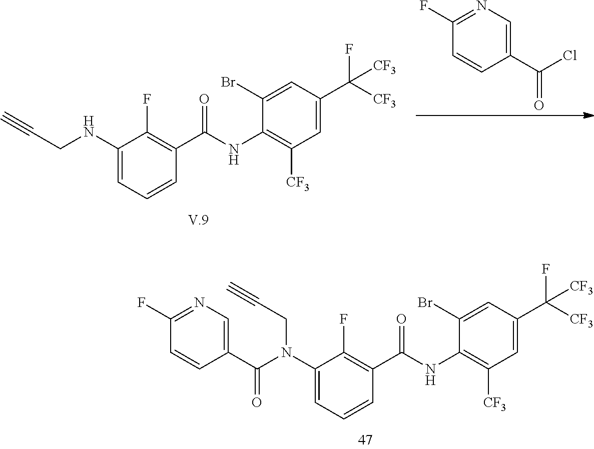 [SMILES: C#CCNc1cccc(C(=O)Nc2c(Br)cc(C(F)(C(F)(F)F)C(F)(F)F)cc2C(F)(F)F)c1F ; O=C(Cl)c1ccc(F)nc1 ; C#CCN(C(=O)c1ccc(F)nc1)c1cccc(C(=O)Nc2c(Br)cc(C(F)(C(F)(F)F)C(F)(F)F)cc2C(F)(F)F)c1F]

0.15 g (0.26 mmol) of an intermediate V.9, 0.06 g (0.40 mmol) of sodium iodide, and 0.06 g (0.38 mmol) of 6-fluoronicotinoyl chloride were added to 10 mL of toluene, and the mixture was heated for reflux. After TLC monitored the completion of the reaction, the reaction product was depressurized and desolventized, and the obtained residue was purified by column chromatography to obtain 0.09 g of a white solid, namely the compound 47. Nuclear magnetism and mass spectrometry data of the compound 47 are as follows:

¹H NMR (600 MHZ, Chloroform-d) δ 8.22 (s, 1H), 8.13 (d, 1H), 8.12-8.08 (m, 1H), 8.03 (d, 1H), 7.94-7.81 (m, 2H), 7.62 (t, 1H), 7.37 (t, 1H), 6.85 (d, 1H), 4.90 (s, 1H), 4.54 (s, 1H), 2.39-2.26 (m, 1H). LC-MS (m/z, ESI): 706.03 (M+H)⁺.

### Example 13: Preparation of Compound 48

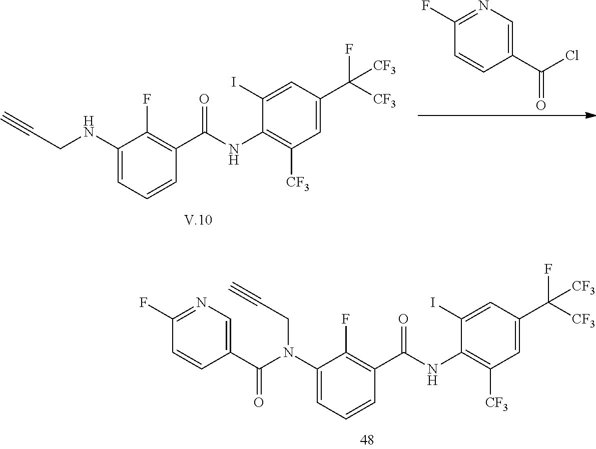 [SMILES: C#CCNc1cccc(C(=O)Nc2c(I)cc(C(F)(C(F)(F)F)C(F)(F)F)cc2C(F)(F)F)c1F ; O=C(Cl)c1ccc(F)nc1 ; C#CCN(C(=O)c1ccc(F)nc1)c1cccc(C(=O)Nc2c(I)cc(C(F)(C(F)(F)F)C(F)(F)F)cc2C(F)(F)F)c1F]

0.15 g (0.24 mmol) of an intermediate V.10, 0.05 g (0.33 mmol) of sodium iodide, and 0.06 g (0.38 mmol) of 6-fluoronicotinoyl chloride were added to 10 mL of toluene, and the mixture was heated for reflux. After TLC monitored the completion of the reaction, the reaction product was depressurized and desolventized, and the obtained residue was purified by column chromatography to obtain 0.09 g of a yellow oily substance, namely the compound 48. Nuclear magnetism and mass spectrometry data of the compound 48 are as follows:

¹H NMR (600 MHZ, Chloroform-d) δ 8.34 (d, 1H), 8.22 (s, 1H), 8.14-7.99 (m, 2H), 7.96-7.90 (m, 1H), 7.86 (s, 1H), 7.69-7.59 (m, 1H), 7.38 (t, 1H), 6.84 (d, 1H), 4.90 (s, 1H), 4.55 (s, 1H), 2.33 (t, 1H). LC-MS (m/z, ESI): 754.05 (M+H)⁺.

Other compounds in General Formulas I-VII of the present invention can be prepared by referring to the above examples.

The physicochemical properties, nuclear magnetism and mass spectrometry data of some compounds of the present invention are as follows:

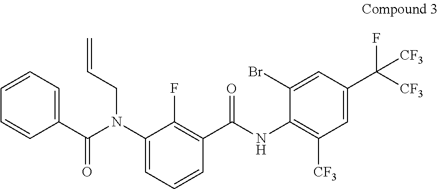 [SMILES: C=CCN(C(=O)c1ccccc1)c1cccc(C(=O)Nc2c(Br)cc(C(F)(C(F)(F)F)C(F)(F)F)cc2C(F)(F)F)c1F]

White solid. ¹H NMR (600 MHz, Chloroform-d) δ 8.12 (d, 1H), 7.98 (t, 1H), 7.89 (d, 1H), 7.46 (t, 1H), 7.42-7.11 (m, 7H), 6.07-5.93 (br s, 1H), 5.27-5.15 (m, 2H), 4.53 (d, 3H). LC-MS (m/z, ESI): 689.10 (M+H)⁺.

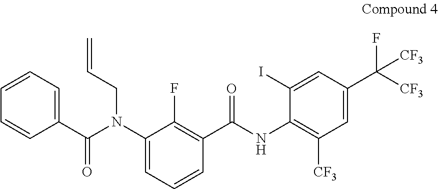 [SMILES: C=CCN(C(=O)c1ccccc1)c1cccc(C(=O)Nc2c(I)cc(C(F)(C(F)(F)F)C(F)(F)F)cc2C(F)(F)F)c1F]

Yellow solid. ¹H NMR (600 MHz, Chloroform-d) δ 8.32 (d, 1H), 7.98 (t, 1H), 7.92 (d, 1H), 7.51-7.44 (m, 1H), 7.43-7.10 (m, 7H), 6.10-5.90 (br s, 1H), 5.28-5.14 (m, 2H), 4.54 (d, 2H). LC-MS (m/z, ESI): 737.07 (M+H)⁺.

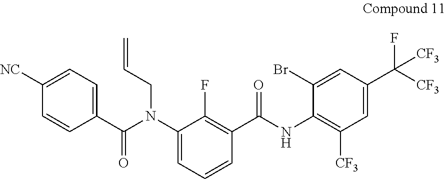 [SMILES: C=CCN(C(=O)c1ccc(C#N)cc1)c1cccc(C(=O)Nc2c(Br)cc(C(F)(C(F)(F)F)C(F)(F)F)cc2C(F)(F)F)c1F]

White solid. ¹H NMR (600 MHZ, Chloroform-d) δ 8.13 (d, 1H), 8.03 (t, 1H), 7.99-7.86 (m, 2H), 7.67-7.37 (m, 5H), 7.30 (t, 1H), 6.07-5.89 (br, 1H), 5.29-5.16 (m, 2H), 4.54 (d, 2H). LC-MS (m/z, ESI): 714.11 (M+H)⁺.

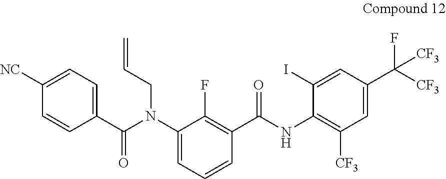 [SMILES: C=CCN(C(=O)c1ccc(C#N)cc1)c1cccc(C(=O)Nc2c(I)cc(C(F)(C(F)(F)F)C(F)(F)F)cc2C(F)(F)F)c1F]

White solid. ¹H NMR (600 MHz, Chloroform-d) δ 8.34 (d, 1H), 8.10-7.90 (m, 3H), 7.61-7.37 (m, 5H), 7.31 (t, 1H), 6.06-5.91 (br, 1H), 5.29-5.17 (m, 2H), 4.54 (d, 2H). LC-MS (m/z, ESI): 762.05 (M+H)⁺.

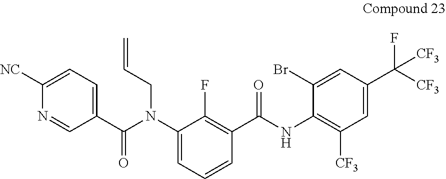 [SMILES: C=CCN(C(=O)c1ccc(C#N)nc1)c1cccc(C(=O)Nc2c(Br)cc(C(F)(C(F)(F)F)C(F)(F)F)cc2C(F)(F)F)c1F]

Oily substance. ¹H NMR (600 MHZ, Chloroform-d) δ 8.62 (s, 1H), 8.13 (d, 1H), 8.06 (t, 1H), 7.97-7.89 (m, 2H), 7.82 (d, 1H), 7.58 (d, 1H), 7.51 (t, 1H), 7.35 (t, 1H), 6.07-5.89 (m, 1H), 5.28-5.21 (m, 2H), 4.55 (d, 2H). LC-MS (m/z, ESI): 715.11 (M+H)⁺.

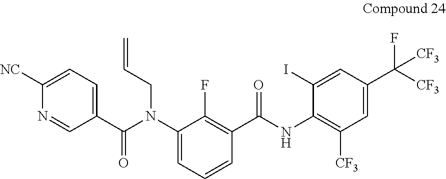 [SMILES: C=CCN(C(=O)c1ccc(C#N)nc1)c1cccc(C(=O)Nc2c(I)cc(C(F)(C(F)(F)F)C(F)(F)F)cc2C(F)(F)F)c1F]

Yellow solid. ¹H NMR (600 MHz, Chloroform-d) δ 8.62 (s, 1H), 8.36-8.31 (m, 1H), 8.07 (t, 1H), 8.01-7.89 (m, 2H), 7.82 (d, 1H), 7.58 (d, 1H), 7.52 (d, 1H), 7.36 (t, 1H), 6.06-5.90 (m, 1H), 5.30-5.19 (m, 2H), 4.56 (d, 2H). LC-MS (m/z, ESI): 763.04 (M+H)⁺.

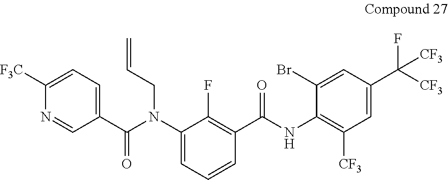 [SMILES: C=CCN(C(=O)c1ccc(C(F)(F)F)nc1)c1cccc(C(=O)Nc2c(Br)cc(C(F)(C(F)(F)F)C(F)(F)F)cc2C(F)(F)F)c1F]

White solid. ¹H NMR (600 MHZ, Chloroform-d) δ 8.64 (s, 1H), 8.15-8.10 (m, 1H), 8.07 (t, 1H), 7.97-7.84 (m, 3H), 7.64-7.50 (m, 2H), 7.36 (t, 1H), 6.07-5.92 (br, 1H), 5.29-5.20 (m, 2H), 4.57 (s, 2H). LC-MS (m/z, ESI): 758.09 (M+H)⁺.

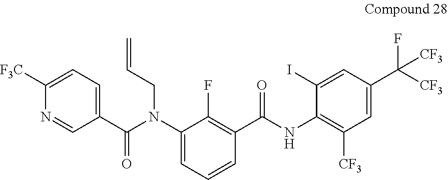 [SMILES: C=CCN(C(=O)c1ccc(C(F)(F)F)nc1)c1cccc(C(=O)Nc2c(I)cc(C(F)(C(F)(F)F)C(F)(F)F)cc2C(F)(F)F)c1F]

White solid. ¹H NMR (600 MHz, Chloroform-d) δ 8.65 (s, 1H), 8.32 (s, 1H), 8.07 (t, 1H), 8.02-7.80 (m, 3H), 7.64-7.50 (m, 2H), 7.37 (t, 1H), 6.07-5.92 (br, 1H), 5.29-5.21 (m, 2H), 4.57 (s, 2H). LC-MS (m/z, ESI): 806.08 (M+H)⁺.

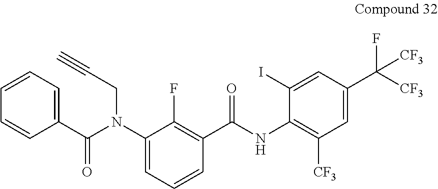 [SMILES: C#CCN(C(=O)c1ccccc1)c1cccc(C(=O)Nc2c(I)cc(C(F)(C(F)(F)F)C(F)(F)F)cc2C(F)(F)F)c1F]

Oily substance. ¹H NMR (600 MHz, Chloroform-d) δ 8.35-8.31 (m, 1H), 8.17-7.96 (m, 2H), 7.95-7.90 (m, 1H), 7.63-7.55 (br, 1H), 7.51-7.04 (m, 6H), 4.93 (s, 1H), 4.52 (s, 1H), 2.30 (t, 1H). LC-MS (m/z, ESI): 735.06 (M+H)⁺.

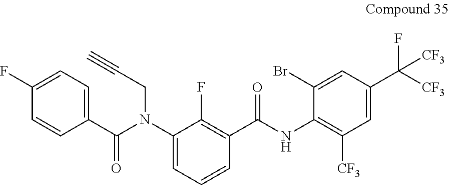 [SMILES: C#CCN(C(=O)c1ccc(F)cc1)c1cccc(C(=O)Nc2c(Br)cc(C(F)(C(F)(F)F)C(F)(F)F)cc2C(F)(F)F)c1F]

White solid. ¹H NMR (600 MHZ, Chloroform-d) δ 8.13 (d, 1H), 8.11-7.99 (m, 2H), 7.93-7.88 (m, 1H), 7.63-7.54 (m, 1H), 7.47-7.35 (m, 2H), 7.32 (t, 1H), 7.02-6.82 (br, 2H), 4.89 (s, 1H), 4.51 (s, 1H), 2.30 (t, 1H). LC-MS (m/z, ESI): 705.04 (M+H)⁺.

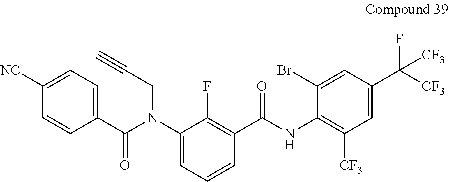 [SMILES: C#CCN(C(=O)c1ccc(C#N)cc1)c1cccc(C(=O)Nc2c(Br)cc(C(F)(C(F)(F)F)C(F)(F)F)cc2C(F)(F)F)c1F]

Oily substance. ¹H NMR (600 MHz, Chloroform-d) δ 8.14 (d, 1H), 8.09 (t, 1H), 8.05-7.95 (br s, 1H), 7.91 (d, 1H), 7.72-7.38 (m, 5H), 7.34 (t, 1H), 4.93 (s, 1H), 4.52 (s, 1H), 2.33 (s, 1H). LC-MS (m/z, ESI): 712.07 (M+H)⁺.

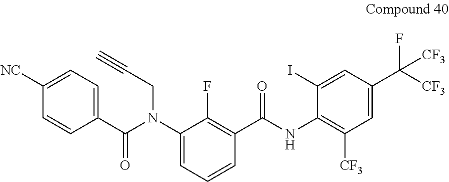 [SMILES: C#CCN(C(=O)c1ccc(C#N)cc1)c1cccc(C(=O)Nc2c(I)cc(C(F)(C(F)(F)F)C(F)(F)F)cc2C(F)(F)F)c1F]

White solid. ¹H NMR (600 MHZ, Chloroform-d) δ 8.34 (d, 1H), 8.19-7.97 (m, 2H), 7.93 (d, 1H), 7.72-7.39 (m, 5H), 7.34 (t, 1H), 4.94 (s, 1H), 4.53 (s, 1H), 2.33 (s, 1H). LC-MS (m/z, ESI): 760.04 (M+H)⁺.

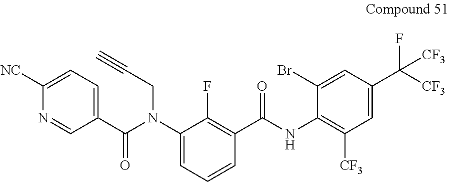 [SMILES: C#CCN(C(=O)c1ccc(C#N)nc1)c1cccc(C(=O)Nc2c(Br)cc(C(F)(C(F)(F)F)C(F)(F)F)cc2C(F)(F)F)c1F]

White solid. ¹H NMR (600 MHz, Chloroform-d) δ 8.64 (s, 1H), 8.16-8.08 (m, 2H), 7.97 (d, 1H), 7.91 (d, 1H), 7.85 (s, 1H), 7.68-7.56 (m, 2H), 7.38 (t, 1H), 4.93 (d, 1H), 4.55 (d, 1H), 2.35 (s, 1H). LC-MS (m/z, ESI): 735.05 (M+Na)⁺.

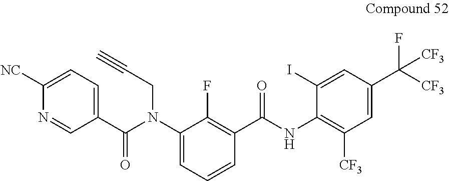 [SMILES: C#CCN(C(=O)c1ccc(C#N)nc1)c1cccc(C(=O)Nc2c(I)cc(C(F)(C(F)(F)F)C(F)(F)F)cc2C(F)(F)F)c1F]

White solid. ¹H NMR (600 MHz, Chloroform-d) δ 8.64 (s, 1H), 8.34 (d, 1H), 8.12 (t, 1H), 8.07-7.96 (m, 1H), 7.93 (d, 1H), 7.84 (s, 1H), 7.70-7.54 (m, 2H), 7.39 (t, 1H), 4.93 (d, 1H), 4.56 (d, 1H), 2.35 (s, 1H). LC-MS (m/z, ESI): 761.00 (M+H)⁺.

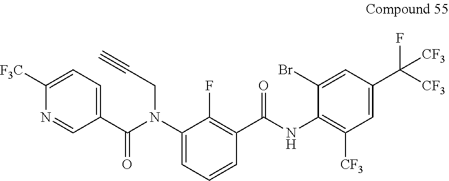 [SMILES: C#CCN(C(=O)c1ccc(C(F)(F)F)nc1)c1cccc(C(=O)Nc2c(Br)cc(C(F)(C(F)(F)F)C(F)(F)F)cc2C(F)(F)F)c1F]

White solid. ¹H NMR (600 MHz, Chloroform-d) δ 8.67 (s, 1H), 8.17-8.07 (m, 2H), 8.03-7.85 (m, 3H), 7.70-7.55 (m, 2H), 7.43-7.34 (m, 1H), 4.93 (d, 1H), 4.58 (d, 1H), 2.35 (s, 1H). LC-MS (m/z, ESI): 756.09 (M+H)⁺.

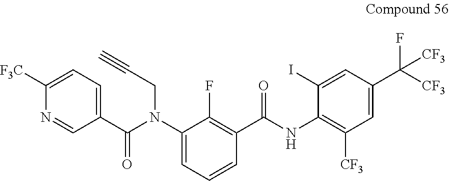 [SMILES: C#CCN(C(=O)c1ccc(C(F)(F)F)nc1)c1cccc(C(=O)Nc2c(I)cc(C(F)(C(F)(F)F)C(F)(F)F)cc2C(F)(F)F)c1F]

White solid. ¹H NMR (600 MHZ, Chloroform-d) δ 8.67 (s, 1H), 8.36-8.31 (m, 1H), 8.12 (t, 1H), 8.00 (d, 1H), 7.95-7.85 (m, 2H), 7.68 (t, 1H), 7.63-7.55 (m, 1H), 7.40 (t, 1H), 4.93 (d, 1H), 4.59 (d, 1H), 2.35 (s, 1H). LC-MS (m/z, ESI): 804.07 (M+H)⁺.

### Bioactivity Determination

### Example 14: Determination of Insecticidal Bioactivity

The insecticidal activity of the compound of the present invention was determined on several insects. A determination method was as follows:

After the compound to be determined was dissolved in a mixed solvent of acetone/methanol (1:1), it was diluted with water containing 0.1% (wt) Tween-80 to a desired concentration.

Mythimna separata, Diamondback moth, Chilo suppressalis, Spodoptera exigua, Myzus persicae, and Franklinella occidentalis were used as targets, and the activity was tested by an Airbrush spray method.

### (1) Determination of Activity Against Mythimna separata

Determination method: maize leaves were cut into 2 cm leaf pieces, and the front and back of each leaf piece were sprayed under an Airbrush spray treatment pressure of 10 psi (approximate to 0.7 kg/cm²) and with a spray volume of 0.5 mL. After drying in the shade, 10 3rd-instar larvae were inoculated for each treatment, and each treatment was repeated for 3 times. After the treatment, the leaf pieces were put into an observation chamber at 25° C. and with a relative humidity of 60% to 70%, and the number of living larvae was investigated 3 days after the administration of the compounds, and the mortality rates were calculated.

Partial test results for Mythimna separata were as follows:

At a dosage of 0.05 mg/L, 3 days after the administration, compounds 3, 4, 7, 8, 11, 12, 19, 20, 23, 24, 27, 28, 31, 32, 35, 36, 39, 40, 47, 48, 51, 52, 55, and 56 had a mortality rate of more than 90% against Mythimna separata.

### (2) Determination of Activity Against Diamondback Moth

Determination method: cabbage leaves were formed into leaf discs having a diameter of 2 cm using a puncher, and the front and back of each leaf disc were sprayed under an Airbrush spray treatment pressure of 10 psi (approximate to 0.7 kg/cm²) and with a spray volume of 0.5 mL. After drying in the shade, 10 3rd-instar larvae were inoculated for each treatment, and each treatment was repeated for 3 times. After the treatment, the leaf discs were placed into an observation chamber at 25° C. and with a relative humidity of 60% to 70%, and the number of living larvae was investigated 3 days after the administration of the compounds, and the mortality rates were calculated.

Partial test results for Diamondback moth were as follows:

At a dosage of 1.25 mg/L, compounds 3, 4, 7, 8, 11, 12, 19, 20, 23, 24, 27, 28, 31, 32, 35, 36, 39, 40, 47, 48, 51, 52, 55, and 56 had a mortality rate of more than 90% against Diamondback moth.

According to the above experimental methods, some compounds of the present invention were further selected for parallel determinations of the activity against Diamondback moth with control compounds KC1, KC2, KC9, and KC10, and experimental results are shown in Table 12.

TABLE 12
Parallel comparative experiments of insecticidal activity of some compounds of the
present invention against Diamondback moth with control compounds KCI, KC2, KC9, and
KC10

| | | Mortality rate (%, 3 days |
|---|---|---|
| Compound | | after the administration) |
| Number | Structural formula | 10 mg/L |
| 3 | 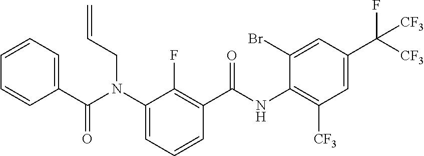 [SMILES: C=CCN(C(=O)c1ccccc1)c1cccc(C(=O)Nc2c(Br)cc(C(F)(C(F)(F)F)C(F)(F)F)cc2C(F)(F)F)c1F] | 100 |
| 4 | 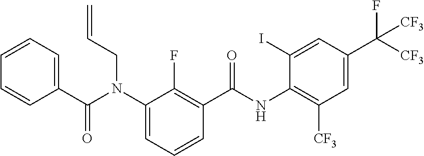 [SMILES: C=CCN(C(=O)c1ccccc1)c1cccc(C(=O)Nc2c(I)cc(C(F)(C(F)(F)F)C(F)(F)F)cc2C(F)(F)F)c1F] | 100 |
| 7 | 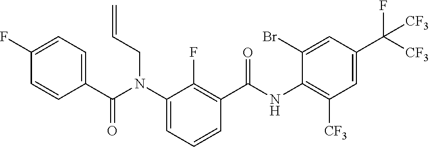 [SMILES: C=CCN(C(=O)c1ccc(F)cc1)c1cccc(C(=O)Nc2c(Br)cc(C(F)(C(F)(F)F)C(F)(F)F)cc2C(F)(F)F)c1F] | 100 |
| 8 | 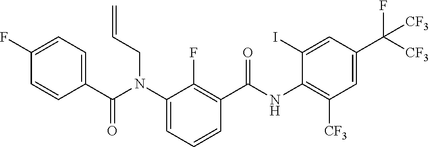 [SMILES: C=CCN(C(=O)c1ccc(F)cc1)c1cccc(C(=O)Nc2c(I)cc(C(F)(C(F)(F)F)C(F)(F)F)cc2C(F)(F)F)c1F] | 100 |
| 11 | 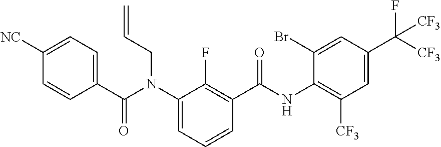 [SMILES: C=CCN(C(=O)c1ccc(C#N)cc1)c1cccc(C(=O)Nc2c(Br)cc(C(F)(C(F)(F)F)C(F)(F)F)cc2C(F)(F)F)c1F] | 100 |
| 12 | 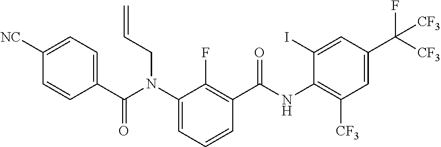 [SMILES: C=CCN(C(=O)c1ccc(C#N)cc1)c1cccc(C(=O)Nc2c(I)cc(C(F)(C(F)(F)F)C(F)(F)F)cc2C(F)(F)F)c1F] | 100 |
| 19 | 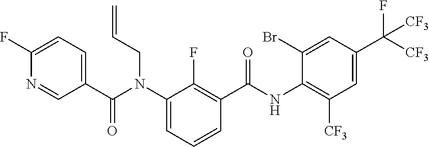 [SMILES: C=CCN(C(=O)c1ccc(F)nc1)c1cccc(C(=O)Nc2c(Br)cc(C(F)(C(F)(F)F)C(F)(F)F)cc2C(F)(F)F)c1F] | 100 |
| 20 | 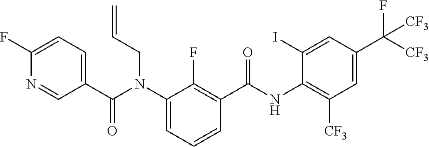 [SMILES: C=CCN(C(=O)c1ccc(F)nc1)c1cccc(C(=O)Nc2c(I)cc(C(F)(C(F)(F)F)C(F)(F)F)cc2C(F)(F)F)c1F] | 100 |
| 23 | 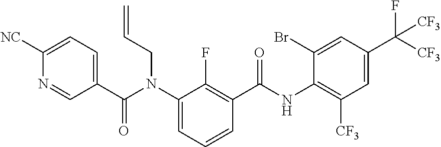 [SMILES: C=CCN(C(=O)c1ccc(C#N)nc1)c1cccc(C(=O)Nc2c(Br)cc(C(F)(C(F)(F)F)C(F)(F)F)cc2C(F)(F)F)c1F] | 100 |
| 24 | 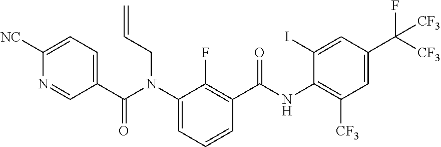 [SMILES: C=CCN(C(=O)c1ccc(C#N)nc1)c1cccc(C(=O)Nc2c(I)cc(C(F)(C(F)(F)F)C(F)(F)F)cc2C(F)(F)F)c1F] | 100 |
| 27 | 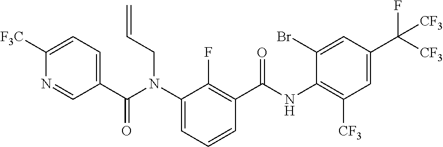 [SMILES: C=CCN(C(=O)c1ccc(C(F)(F)F)nc1)c1cccc(C(=O)Nc2c(Br)cc(C(F)(C(F)(F)F)C(F)(F)F)cc2C(F)(F)F)c1F] | 100 |
| 28 | 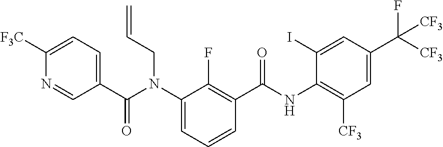 [SMILES: C=CCN(C(=O)c1ccc(C(F)(F)F)nc1)c1cccc(C(=O)Nc2c(I)cc(C(F)(C(F)(F)F)C(F)(F)F)cc2C(F)(F)F)c1F] | 100 |
| 31 | 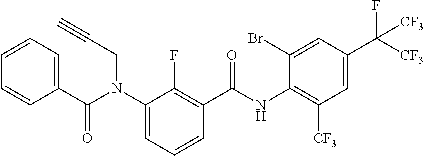 [SMILES: C#CCN(C(=O)c1ccccc1)c1cccc(C(=O)Nc2c(Br)cc(C(F)(C(F)(F)F)C(F)(F)F)cc2C(F)(F)F)c1F] | 100 |
| 32 | 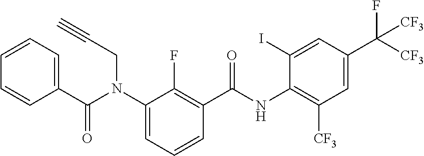 [SMILES: C#CCN(C(=O)c1ccccc1)c1cccc(C(=O)Nc2c(I)cc(C(F)(C(F)(F)F)C(F)(F)F)cc2C(F)(F)F)c1F] | 100 |
| 35 | 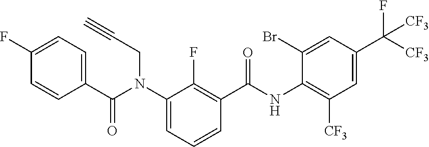 [SMILES: C#CCN(C(=O)c1ccc(F)cc1)c1cccc(C(=O)Nc2c(Br)cc(C(F)(C(F)(F)F)C(F)(F)F)cc2C(F)(F)F)c1F] | 100 |
| 36 | 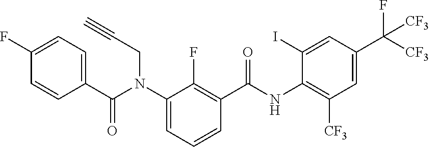 [SMILES: C#CCN(C(=O)c1ccc(F)cc1)c1cccc(C(=O)Nc2c(I)cc(C(F)(C(F)(F)F)C(F)(F)F)cc2C(F)(F)F)c1F] | 100 |
| 39 | 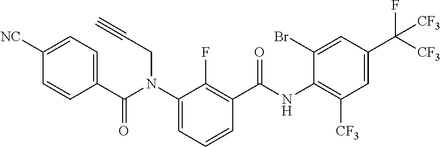 [SMILES: C#CCN(C(=O)c1ccc(C#N)cc1)c1cccc(C(=O)Nc2c(Br)cc(C(F)(C(F)(F)F)C(F)(F)F)cc2C(F)(F)F)c1F] | 100 |
| 40 | 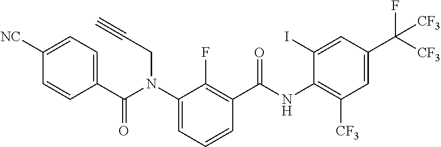 [SMILES: C#CCN(C(=O)c1ccc(C#N)cc1)c1cccc(C(=O)Nc2c(I)cc(C(F)(C(F)(F)F)C(F)(F)F)cc2C(F)(F)F)c1F] | 100 |
| 47 | 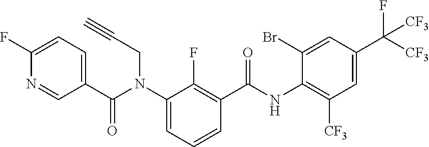 [SMILES: C#CCN(C(=O)c1ccc(F)nc1)c1cccc(C(=O)Nc2c(Br)cc(C(F)(C(F)(F)F)C(F)(F)F)cc2C(F)(F)F)c1F] | 100 |
| 48 | 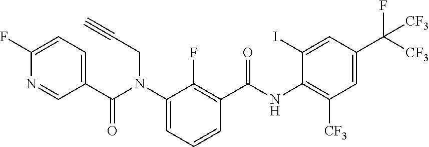 [SMILES: C#CCN(C(=O)c1ccc(F)nc1)c1cccc(C(=O)Nc2c(I)cc(C(F)(C(F)(F)F)C(F)(F)F)cc2C(F)(F)F)c1F] | 100 |
| 51 | 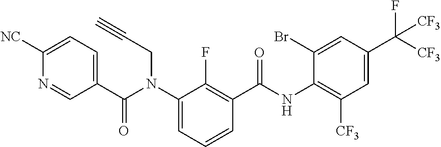 [SMILES: C#CCN(C(=O)c1ccc(C#N)nc1)c1cccc(C(=O)Nc2c(Br)cc(C(F)(C(F)(F)F)C(F)(F)F)cc2C(F)(F)F)c1F] | 100 |
| 52 | 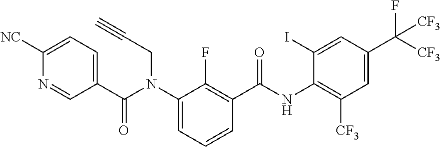 [SMILES: C#CCN(C(=O)c1ccc(C#N)nc1)c1cccc(C(=O)Nc2c(I)cc(C(F)(C(F)(F)F)C(F)(F)F)cc2C(F)(F)F)c1F] | 100 |
| 55 | 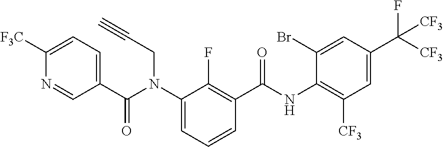 [SMILES: C#CCN(C(=O)c1ccc(C(F)(F)F)nc1)c1cccc(C(=O)Nc2c(Br)cc(C(F)(C(F)(F)F)C(F)(F)F)cc2C(F)(F)F)c1F] | 100 |
| 56 | 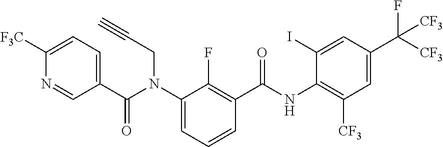 [SMILES: C#CCN(C(=O)c1ccc(C(F)(F)F)nc1)c1cccc(C(=O)Nc2c(I)cc(C(F)(C(F)(F)F)C(F)(F)F)cc2C(F)(F)F)c1F] | 100 |
| KC1 | 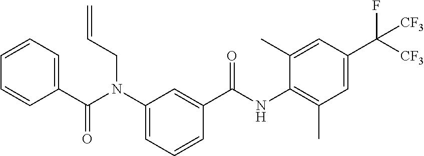 [SMILES: C=CCN(C(=O)c1ccccc1)c1cccc(C(=O)Nc2c(C)cc(C(F)(C(F)(F)F)C(F)(F)F)cc2C)c1] | 0 |
| KC2 | 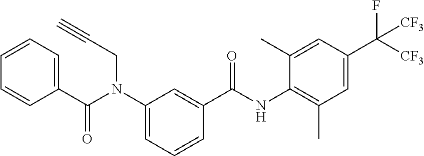 [SMILES: C#CCN(C(=O)c1ccccc1)c1cccc(C(=O)Nc2c(C)cc(C(F)(C(F)(F)F)C(F)(F)F)cc2C)c1] | 0 |
| KC9 | 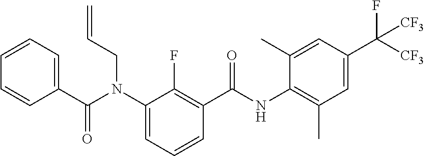 [SMILES: C=CCN(C(=O)c1ccccc1)c1cccc(C(=O)Nc2c(C)cc(C(F)(C(F)(F)F)C(F)(F)F)cc2C)c1F] | 100 |
| KC10 | 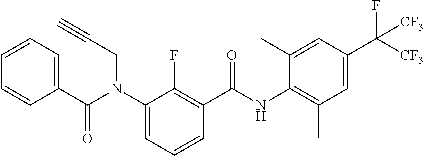 [SMILES: C#CCN(C(=O)c1ccccc1)c1cccc(C(=O)Nc2c(C)cc(C(F)(C(F)(F)F)C(F)(F)F)cc2C)c1F] | 100 |

Note: KC9 and KC10 in the table are control compounds additionally provided in the present application, which can be obtained by referring to the method in Example 10 of the present invention. The raw materials are all those that may be prepared or purchased according to the methods in the examples of the present invention, or may be prepared according to conventional methods.

By comparing the compounds of the present invention with the control compounds KC1, KC2, KC9, and KC10, comparing KC9 with KC1, and comparing KC10 with KC2, it may be concluded that: compared to existing technologies, the compound of the present invention has unexpectedly high insecticidal activity and substantial progress.

According to the above experimental methods, the compounds 3, 7, 8, 19, 20, 31, 35, 36, 47, and 48 of the present invention were further selected for parallel determinations of the activity against Diamondback moth with control compounds KC3, KC4, KC5, KC6, KC7, and KC8, and experimental results are shown in Table 13.

TABLE 13
Parallel comparative experiments of insecticidal activity of some compounds of the
present invention against Diamondback moth with control compounds

| | | Mortality rate (%, 3 days after the | |
|---|---|---|---|
| | | administration) | |
| Compound | | 0.313 | 0.156 |
| Number | Structural formula | mg/L | mg/L |
| 3 | 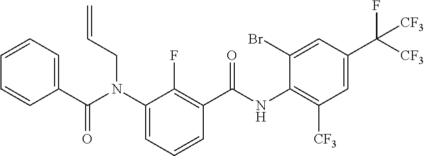 [SMILES: C=CCN(C(=O)c1ccccc1)c1cccc(C(=O)Nc2c(Br)cc(C(F)(C(F)(F)F)C(F)(F)F)cc2C(F)(F)F)c1F] | 100 | 100 |
| 31 | 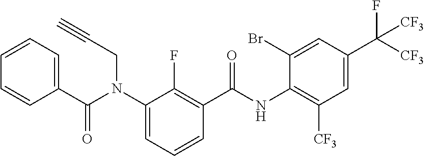 [SMILES: C#CCN(C(=O)c1ccccc1)c1cccc(C(=O)Nc2c(Br)cc(C(F)(C(F)(F)F)C(F)(F)F)cc2C(F)(F)F)c1F] | 100 | 100 |
| KC3 | 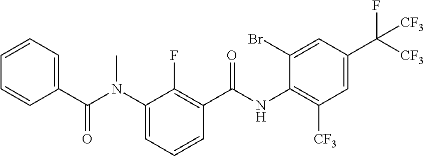 [SMILES: CN(C(=O)c1ccccc1)c1cccc(C(=O)Nc2c(Br)cc(C(F)(C(F)(F)F)C(F)(F)F)cc2C(F)(F)F)c1F] | 93.33 | 50 |
| 7 | 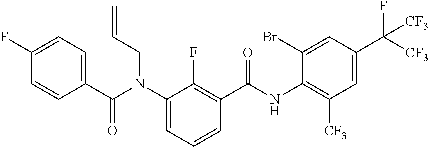 [SMILES: C=CCN(C(=O)c1ccc(F)cc1)c1cccc(C(=O)Nc2c(Br)cc(C(F)(C(F)(F)F)C(F)(F)F)cc2C(F)(F)F)c1F] | 100 | 100 |
| 35 | 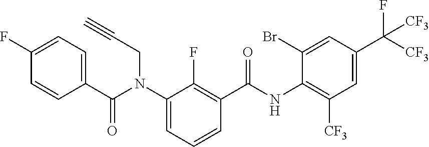 [SMILES: C#CCN(C(=O)c1ccc(F)cc1)c1cccc(C(=O)Nc2c(Br)cc(C(F)(C(F)(F)F)C(F)(F)F)cc2C(F)(F)F)c1F] | 100 | 100 |
| KC4 | 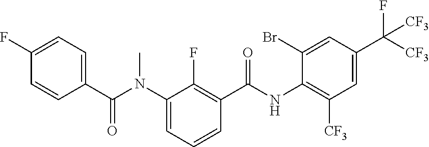 [SMILES: CN(C(=O)c1ccc(F)cc1)c1cccc(C(=O)Nc2c(Br)cc(C(F)(C(F)(F)F)C(F)(F)F)cc2C(F)(F)F)c1F] | 100 | 60 |
| 8 | 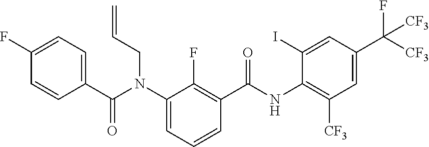 [SMILES: C=CCN(C(=O)c1ccc(F)cc1)c1cccc(C(=O)Nc2c(I)cc(C(F)(C(F)(F)F)C(F)(F)F)cc2C(F)(F)F)c1F] | 100 | 100 |
| 36 | 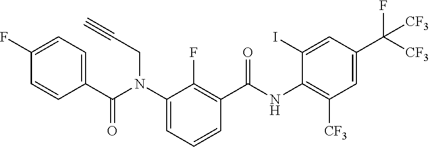 [SMILES: C#CCN(C(=O)c1ccc(F)cc1)c1cccc(C(=O)Nc2c(I)cc(C(F)(C(F)(F)F)C(F)(F)F)cc2C(F)(F)F)c1F] | 100 | 100 |
| KC5 | 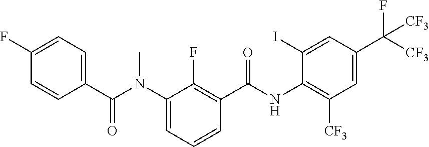 [SMILES: CN(C(=O)c1ccc(F)cc1)c1cccc(C(=O)Nc2c(I)cc(C(F)(C(F)(F)F)C(F)(F)F)cc2C(F)(F)F)c1F] | 100 | 73.33 |
| 19 | 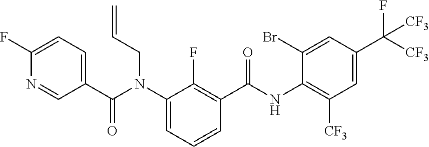 [SMILES: C=CCN(C(=O)c1ccc(F)nc1)c1cccc(C(=O)Nc2c(Br)cc(C(F)(C(F)(F)F)C(F)(F)F)cc2C(F)(F)F)c1F] | 100 | 100 |
| 47 | 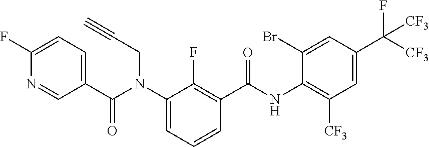 [SMILES: C#CCN(C(=O)c1ccc(F)nc1)c1cccc(C(=O)Nc2c(Br)cc(C(F)(C(F)(F)F)C(F)(F)F)cc2C(F)(F)F)c1F] | 100 | 100 |
| KC7 | 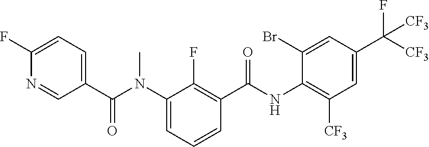 [SMILES: CN(C(=O)c1ccc(F)nc1)c1cccc(C(=O)Nc2c(Br)cc(C(F)(C(F)(F)F)C(F)(F)F)cc2C(F)(F)F)c1F] | 100 | 56.67 |
| 20 | 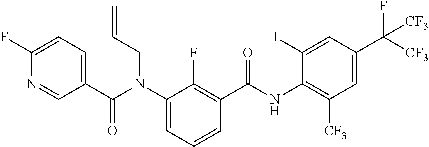 [SMILES: C=CCN(C(=O)c1ccc(F)nc1)c1cccc(C(=O)Nc2c(I)cc(C(F)(C(F)(F)F)C(F)(F)F)cc2C(F)(F)F)c1F] | 100 | 100 |
| 48 | 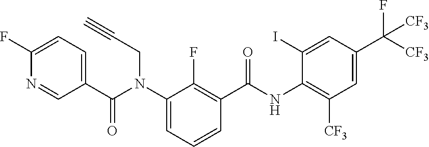 [SMILES: C#CCN(C(=O)c1ccc(F)nc1)c1cccc(C(=O)Nc2c(I)cc(C(F)(C(F)(F)F)C(F)(F)F)cc2C(F)(F)F)c1F] | 100 | 100 |
| KC6 | 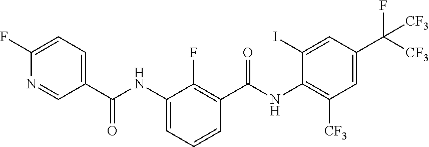 [SMILES: O=C(Nc1cccc(C(=O)Nc2c(I)cc(C(F)(C(F)(F)F)C(F)(F)F)cc2C(F)(F)F)c1F)c1ccc(F)nc1] | 90 | 46.67 |
| KC8 | 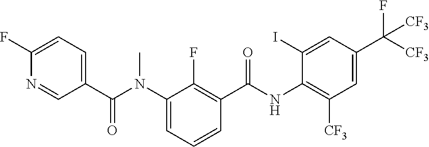 [SMILES: CN(C(=O)c1ccc(F)nc1)c1cccc(C(=O)Nc2c(I)cc(C(F)(C(F)(F)F)C(F)(F)F)cc2C(F)(F)F)c1F] | 86.67 | 53.33 |

As shown in Table 13, by comparing the compounds 3 and 31 with the control compound KC3. comparing the compounds 7 and 35 with the control compound KC4, comparing the compounds 8 and 36 with the control compound KC5, comparing the compounds 19 and 47 with the control compound KC7, and comparing the compounds 20 and 48 with the control compounds KC6 and KC8, it may be seen that: in the examples of the present invention, allyl and propargyl are introduced into Re in the compound in General Formula I, so the compound with better insecticidal effect is obtained compared to existing technologies.

### (3) Determination of Activity Against Chilo suppressalis

Determination method: 1) Preparation of rice seedlings: rice was cultured in a plastic small cup with a diameter of 4.5 cm and a height of 4 cm in a constant temperature room (the temperature was 26-28° C., the relative humidity was about 60-80%, and the illumination was 16hL:8hD), when the rice grown to a 4-5 leaf stage, robust and uniformly grown rice seedlings were selected for pharmaceutical treatment, and 3 replicates were set for each treatment. 2) Preparation of test insects: 3rd-instar larvae of Chilo suppressalis were continuously reared indoors. 3) Rice stems were sprayed and insects were introduced. A spray method was used to treat whole rice seedlings with uniform spray, and each treatment was 15 mL. Blank controls were treated firstly, and then the above operation was repeated in an order of experimental concentrations from low to high. After the spray treatment of the rice seedlings, they were placed in the shade to air-dry drug liquid, about 5 cm of the stem from a basal part of the stem was cut and fed to the test insects. A glass culture dish with a diameter of 90 mm was prepared, filter paper was placed on the bottom of the dish, then water was added to moisturize. About 5 rice stems were placed in each dish, 10 larvae were introduced, and the culture dish was closed with non-woven fabric and placed in the constant-temperature room for culture. 3 days after the administration, the number of residual live insects was investigated.

Partial test results of Chilo suppressalis were as follows:

At a dosage of 0.625 mg/L, the compounds 3, 4, 7, 8, 11, 12, 19, 20, 23, 24, 27, 28, 31, 32, 35, 36, 39, 40, 47, 48, 51, 52, 55, and 56 had a mortality rate of more than 90% against Chilo suppressalis.

### (4) Determination of Activity Against Spodoptera exigua

Determination method: the activity was tested by using a leaf disc dipping feeding method. Leaf discs were dipped in a drug solution for 10 s, and placed in a culture dish after being air-dried, there were 4 discs in each dish, and a filter paper was placed in the culture dish for moisture retention. 10 test insects of Spodoptera exigua were introduced to each dish, with 3 replicates.

They were placed in a light incubator for culture at a temperature of 25° C. and an illumination of 14hL:10hD. On the 1st, 2nd, and 3rd day after the administration, the number of dead Spodoptera exigua was investigated and the mortality rate was calculated.

Test results of Spodoptera exigua were as follows:

At a dosage of 0.625 mg/L, 3 days after the administration, the compounds 3, 4, 7, 8, 11, 12, 19, 20, 23, 24, 27, 28, 31, 32, 35, 36, 39, 40, 47, 48, 51, 52, 55, and 56 had a mortality rate of more than 90% against Spodoptera exigua.

### (5) Determination of Activity Against Myzus persicae

Determination method: a culture dish with a diameter of 6 cm was taken, the bottom of the dish was covered by a layer of filter paper, and an appropriate amount of tap water was added to moisturize the paper. Cabbage leaves with an appropriate size (the diameter was about 3 cm) and 15-30 aphids grown were cut from cabbage plants used to culture Myzus persicae, winged aphids and aphids on the front of the leave were removed, and it was placed in the culture dish while the back of the leave was upward. The pressure of airbrush spray treatment was 10 psi (about 0.7 kg/cm²), the liquid spray volume was 0.5 mL, and there were 3 replicates for each treatment. After the treatment, the leave was put into an observation room for culture at 25° C. and a relative humidity of 60-70%. After 48 h, the number of live insects was investigated and the mortality rate was calculated.

Test results for Myzus persicae were as follows:

At a dosage of 50 mg/L, the compounds 3, 4, 7, 8, 11, 12, 19, 20, 23, 24, 27, 28, 31, 32, 35, 36, 39, 40, 47, 48, 51, 52, 55, and 56 had a mortality rate of more than 90% against Myzus persicae.

### (6) Determination of Activity Against Franklinella occidentalis

Determination method: fresh bean leaves cultivated in a greenhouse were selected, a hand-held Airbrush sprayer was used to spray uniformly, each treatment was 1 mL, and after being air-dried naturally, the leaves were placed in a finger-shaped tube. Neat and healthy Franklinella occidentalis nymphs were introduced, there were 15 nymphs for each treatment, 3 replicates were set for the experiment, and water treatment was set as a blank control. After the treatment, the leaves were put into an indoor culture room for culture at 24° C., a relative humidity of 60-70%, and natural light. After 72 h, the number of live insects was investigated and the mortality rate was calculated.

Test results for Franklinella occidentalis were as follows:

At a dosage of 100 mg/L, the compounds 3, 4, 7, 8, 11, 12, 19, 20, 23, 24, 27, 28, 31, 32, 35, 36, 39, 40, 47, 48, 51, 52, 55, and 56 had a mortality rate of more than 90% against Franklinella occidentalis.

According to the above experimental methods, the compounds 3, 7, 8, 19, 20, 31, 35, 36, 47, and 48 of the present invention were further selected for parallel determinations of the activity against Franklinella occidentalis nymphs with control compounds KC3, KC4, KC5, KC6, KC7, and KC8, and experimental results are shown in Table 14.

TABLE 14
Parallel comparative experiments of insecticidal activity of some compounds of the
present invention against Franklinella occidentalis nymphs with control compounds

| | | Mortality rate (%, 3 days after the | |
|---|---|---|---|
| Compound | | administration) | |
| Number | Structural formula | 100 mg/L | 10 mg/L |
| 3 | 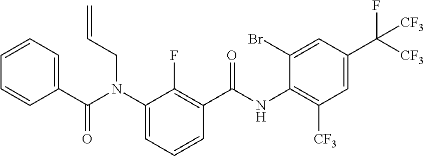 [SMILES: C=CCN(C(=O)c1ccccc1)c1cccc(C(=O)Nc2c(Br)cc(C(F)(C(F)(F)F)C(F)(F)F)cc2C(F)(F)F)c1F] | 100 | 93.33 |
| 31 | 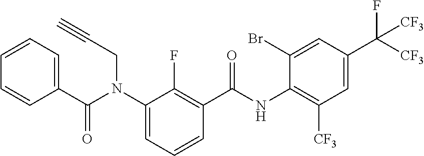 [SMILES: C#CCN(C(=O)c1ccccc1)c1cccc(C(=O)Nc2c(Br)cc(C(F)(C(F)(F)F)C(F)(F)F)cc2C(F)(F)F)c1F] | 100 | 97.78 |
| KC3 | 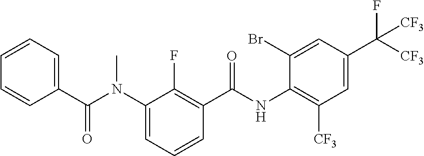 [SMILES: CN(C(=O)c1ccccc1)c1cccc(C(=O)Nc2c(Br)cc(C(F)(C(F)(F)F)C(F)(F)F)cc2C(F)(F)F)c1F] | 57.78 | 31.11 |
| 7 | 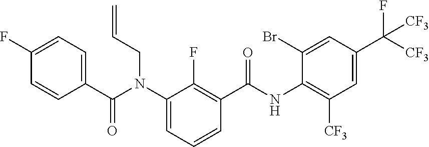 [SMILES: C=CCN(C(=O)c1ccc(F)cc1)c1cccc(C(=O)Nc2c(Br)cc(C(F)(C(F)(F)F)C(F)(F)F)cc2C(F)(F)F)c1F] | 100 | 91.11 |
| 35 | 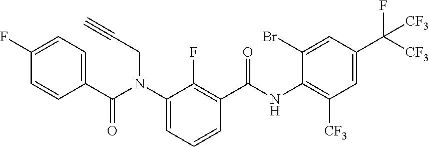 [SMILES: C#CCN(C(=O)c1ccc(F)cc1)c1cccc(C(=O)Nc2c(Br)cc(C(F)(C(F)(F)F)C(F)(F)F)cc2C(F)(F)F)c1F] | 100 | 100 |
| KC4 | 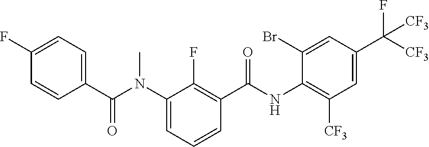 [SMILES: CN(C(=O)c1ccc(F)cc1)c1cccc(C(=O)Nc2c(Br)cc(C(F)(C(F)(F)F)C(F)(F)F)cc2C(F)(F)F)c1F] | 62.22 | 40.00 |
| 8 | 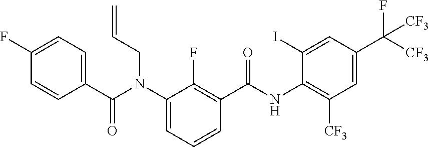 [SMILES: C=CCN(C(=O)c1ccc(F)cc1)c1cccc(C(=O)Nc2c(I)cc(C(F)(C(F)(F)F)C(F)(F)F)cc2C(F)(F)F)c1F] | 100 | 88.89 |
| 36 | 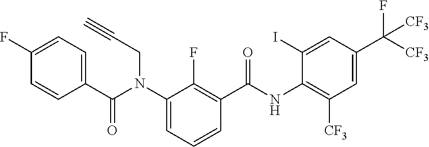 [SMILES: C#CCN(C(=O)c1ccc(F)cc1)c1cccc(C(=O)Nc2c(I)cc(C(F)(C(F)(F)F)C(F)(F)F)cc2C(F)(F)F)c1F] | 100 | 95.56 |
| KC5 | 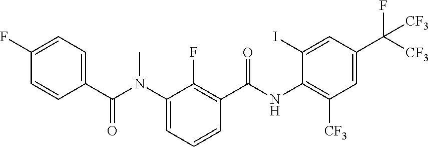 [SMILES: CN(C(=O)c1ccc(F)cc1)c1cccc(C(=O)Nc2c(I)cc(C(F)(C(F)(F)F)C(F)(F)F)cc2C(F)(F)F)c1F] | 51.11 | 28.89 |
| 19 | 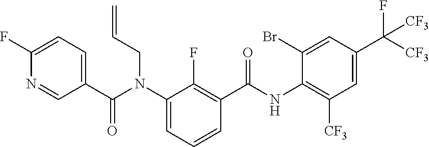 [SMILES: C=CCN(C(=O)c1ccc(F)nc1)c1cccc(C(=O)Nc2c(Br)cc(C(F)(C(F)(F)F)C(F)(F)F)cc2C(F)(F)F)c1F] | 100 | 100 |
| 47 | 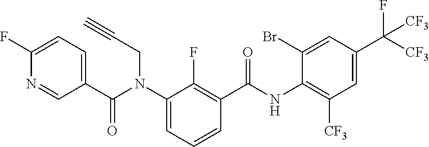 [SMILES: C#CCN(C(=O)c1ccc(F)nc1)c1cccc(C(=O)Nc2c(Br)cc(C(F)(C(F)(F)F)C(F)(F)F)cc2C(F)(F)F)c1F] | 100 | 100 |
| KC7 | 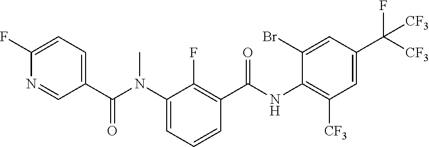 [SMILES: CN(C(=O)c1ccc(F)nc1)c1cccc(C(=O)Nc2c(Br)cc(C(F)(C(F)(F)F)C(F)(F)F)cc2C(F)(F)F)c1F] | 80.00 | 53.33 |
| 20 | 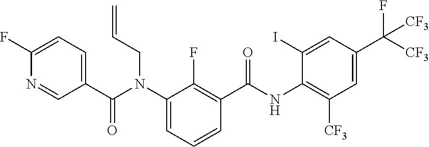 [SMILES: C=CCN(C(=O)c1ccc(F)nc1)c1cccc(C(=O)Nc2c(I)cc(C(F)(C(F)(F)F)C(F)(F)F)cc2C(F)(F)F)c1F] | 100 | 100 |
| 48 | 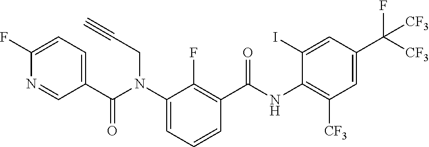 [SMILES: C#CCN(C(=O)c1ccc(F)nc1)c1cccc(C(=O)Nc2c(I)cc(C(F)(C(F)(F)F)C(F)(F)F)cc2C(F)(F)F)c1F] | 100 | 100 |
| KC6 | 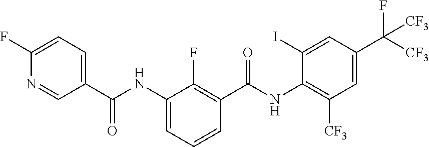 [SMILES: O=C(Nc1cccc(C(=O)Nc2c(I)cc(C(F)(C(F)(F)F)C(F)(F)F)cc2C(F)(F)F)c1F)c1ccc(F)nc1] | 77.78 | 57.78 |
| KC8 | 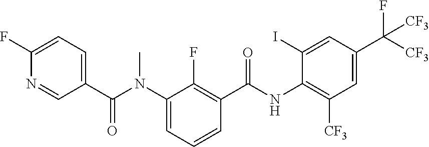 [SMILES: CN(C(=O)c1ccc(F)nc1)c1cccc(C(=O)Nc2c(I)cc(C(F)(C(F)(F)F)C(F)(F)F)cc2C(F)(F)F)c1F] | 82.22 | 53.33 |

As shown in Table 14. by comparing the compounds 3 and 31 with the control compound KC3. comparing the compounds 7 and 35 with the control compound KC4, comparing the compounds 8 and 36 with the control compound KC5, comparing the compounds 19 and 47 with the control compound KC7, and comparing the compounds 20 and 48 with the control compounds KC6 and KC8, it may be seen that: in the examples of the present invention, allyl and propargyl are introduced into Re in the compound in General Formula I, so the compound with better insecticidal effect is obtained compared to existing technologies.

### Example 15: Insecticidal Experiment on Cat Flea

4 mg of the compound to be determined was dissolved in 40 mL of acetone to obtain an acetone solution with a concentration of 100 mg/L. 400 μL of the drug solution was applied on the bottom and sides of a culture dish with an inner diameter of 5.3 cm, then after acetone was evaporated, a thin film of the compound of the present invention was made on the inner wall of the culture dish. The inner wall of the culture dish used was 40 cm², and the treatment dosage was 1 μg/cm². 10 adult cat fleas (mixed male and female) were put into the dish, and it was stored in a constant-temperature room at 25° C. after being covered. The number of dead insects after 72 h was checked and the mortality rate was calculated. The experiment was repeated for 3 times. Test result: the compounds 3, 4, 7, 8, 11, 12, 19, 20, 23, 24, 27, 28, 31, 32, 35, 36, 39, 40, 47, 48, 51, 52, 55, and 56 showed a mortality rate of more than 90%.

### Example 16: Insecticidal Experiment on American Dog Tick

4 mg of the compound to be determined was dissolved in 40 mL of acetone to obtain an acetone solution with a concentration of 100 mg/L. 400 μL of the drug solution was applied on the bottom and sides of a culture dish with an inner diameter of 5.3 cm, then after acetone was evaporated, a thin film of the compound of the present invention was made on the inner wall of the culture dish. The inner wall of the culture dish used is 40 cm², and the treatment dosage was 1 μg/cm². 10 1st-nymphs (mixed male and female) of the American dog tick were put into the dish, 2 culture dishes were merged, a junction portion were sealed with an adhesive tape to prevent escape, and the dishes were stored in a constant-temperature room at 25° C. The number of dead insects after 24 h was checked and the mortality rate was calculated. The experiment was repeated for 3 times. Test result: the compounds 3, 4, 7, 8, 11, 12, 19, 20, 23, 24, 27, 28, 31, 32, 35, 36, 39, 40, 47, 48, 51, 52, 55, and 56 showed a mortality rate of more than 90%.

1. An amide compound shown in General Formula I: 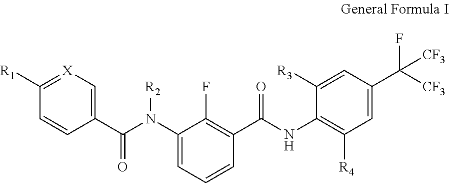
in General Formula I:
R₁ is selected from hydrogen, fluorine, cyano, trifluoromethyl or difluoromethyl;
R₂ is selected from allyl or propargyl;
R₃ selected from halogen;
R₄ is selected from halogen, C₁-C₃ haloalkyl, or C₁-C₃ haloalkoxy; and
X is selected from CH or N.

2. The amide compound according to claim 1, wherein in General Formula I,
R₁ is selected from hydrogen, fluorine, cyano, trifluoromethyl or difluoromethyl;
R₂ is selected from allyl or propargyl;
R₃ is selected from bromine or iodine;
R₄ is selected from bromine, iodine, trifluoromethyl or difluoromethoxy; and
X is selected from CH or N.

3. The amide compound according to claim 2, wherein the amide compound is selected from: compounds in Table 1;
the compounds in Table 1 have the structure shown in General Formula I, and R₁, X, R₂, R₃, and R₄ are shown in Table 1:
TABLE 1

| Compound | | | | | |
|---|---|---|---|---|---|
| Number | R₁ | X | R₂ | R₃ | R₄ |
| 1 | H | CH | Allyl | Br | Br |
| 2 | H | CH | Ally1 | Br | I |
| 3 | H | CH | Allyl | Br | CF₃ |
| 4 | H | CH | Allyl | I | CF₃ |
| 5 | F | CH | Allyl | Br | Br |
| 6 | F | CH | Allyl | Br | I |
| 7 | F | CH | Allyl | Br | CF₃ |
| 8 | F | CH | Allyl | I | CF₃ |
| 9 | CN | CH | Allyl | Br | Br |
| 10 | CN | CH | Allyl | Br | I |
| 11 | CN | CH | Ally1 | Br | CF₃ |
| 12 | CN | CH | Allyl | I | CF₃ |
| 13 | CF₃ | CH | Allyl | Br | Br |
| 14 | CF₃ | CH | Allyl | Br | I |
| 15 | CF₃ | CH | Allyl | Br | CF₃ |
| 16 | CF₃ | CH | Allyl | I | CF₃ |
| 17 | F | N | Allyl | Br | Br |
| 18 | F | N | Allyl | Br | I |
| 19 | F | N | Allyl | Br | CF₃ |
| 20 | F | N | Allyl | I | CF₃ |
| 21 | CN | N | Ally1 | Br | Br |
| 22 | CN | N | Allyl | Br | I |
| 23 | CN | N | Allyl | Br | CF₃ |
| 24 | CN | N | Allyl | I | CF₃ |
| 25 | CF₃ | N | Allyl | Br | Br |
| 26 | CF₃ | N | Allyl | Br | I |
| 27 | CF₃ | N | Allyl | Br | CF₃ |
| 28 | CF₃ | N | Allyl | I | CF₃ |
| 29 | H | CH | Propargyl | Br | Br |
| 30 | H | CH | Propargyl | Br | I |
| 31 | H | CH | Propargyl | Br | CF₃ |
| 32 | H | CH | Propargyl | I | CF₃ |
| 33 | F | CH | Propargyl | Br | Br |
| 34 | F | CH | Propargyl | Br | I |
| 35 | F | CH | Propargyl | Br | CF₃ |
| 36 | F | CH | Propargyl | I | CF₃ |
| 37 | CN | CH | Propargyl | Br | Br |
| 38 | CN | CH | Propargyl | Br | I |
| 39 | CN | CH | Propargyl | Br | CF₃ |
| 40 | CN | CH | Propargyl | I | CF₃ |
| 41 | CF₃ | CH | Propargyl | Br | Br |
| 42 | CF₃ | CH | Propargyl | Br | I |
| 43 | CF₃ | CH | Propargyl | Br | CF₃ |
| 44 | CF₃ | CH | Propargyl | I | CF₃ |
| 45 | F | N | Propargyl | Br | Br |
| 46 | F | N | Propargyl | Br | I |
| 47 | F | N | Propargyl | Br | CF₃ |
| 48 | F | N | Propargyl | I | CF₃ |
| 49 | CN | N | Propargyl | Br | Br |
| 50 | CN | N | Propargyl | Br | I |
| 51 | CN | N | Propargyl | Br | CF₃ |
| 52 | CN | N | Propargyl | I | CF₃ |
| 53 | CF₃ | N | Propargyl | Br | Br |
| 54 | CF₃ | N | Propargyl | Br | I |
| 55 | CF₃ | N | Propargyl | Br | CF₃ |
| 56 | CF₃ | N | Propargyl | I | CF₃ |
| 57 | H | CH | Ally1 | Br | OCHF₂ |
| 58 | H | CH | Allyl | I | OCHF₂ |
| 59 | F | CH | Allyl | Br | OCHF₂ |
| 60 | F | CH | Allyl | I | OCHF₂ |
| 61 | CN | CH | Allyl | Br | OCHF₂ |
| 62 | CN | CH | Allyl | I | OCHF₂ |
| 63 | CF₃ | CH | Allyl | Br | OCHF₂ |
| 64 | CF₃ | CH | Allyl | I | OCHF₂ |
| 65 | F | N | Allyl | Br | OCHF₂ |
| 66 | F | N | Ally1 | I | OCHF₂ |
| 67 | CN | N | Allyl | Br | OCHF₂ |
| 68 | CN | N | Allyl | I | OCHF₂ |
| 69 | CF₃ | N | Ally1 | Br | OCHF₂ |
| 70 | CF₃ | N | Allyl | I | OCHF₂ |
| 71 | H | CH | Propargyl | Br | OCHF₂ |
| 72 | H | CH | Propargyl | I | OCHF₂ |
| 73 | F | CH | Propargyl | Br | OCHF₂ |
| 74 | F | CH | Propargyl | I | OCHF₂ |
| 75 | CN | CH | Propargyl | Br | OCHF₂ |
| 76 | CN | CH | Propargyl | I | OCHF₂ |
| 77 | CF₃ | CH | Propargyl | Br | OCHF₂ |
| 78 | CF₃ | CH | Propargyl | I | OCHF₂ |
| 79 | F | N | Propargyl | Br | OCHF₂ |
| 80 | F | N | Propargyl | I | OCHF₂ |
| 81 | CN | N | Propargyl | Br | OCHF₂ |
| 82 | CN | N | Propargyl | I | OCHF₂ |
| 83 | CF₃ | N | Propargyl | Br | OCHF₂ |
| 84 | CF₃ | N | Propargyl | I | OCHF₂ |

4. The amide compound according to claim 3, wherein the amide compound is selected from: compounds in Table 2;
the compounds in Table 2 have the structure shown in General Formula I, and R₁, X, R₂, R₃, and R₄ are shown in Table 2:
TABLE 2

| Compound | | | | | |
|---|---|---|---|---|---|
| Number | R₁ | X | R₂ | R₃ | R₄ |
| 1 | H | CH | Allyl | Br | Br |
| 2 | H | CH | Allyl | Br | I |
| 3 | H | CH | Allyl | Br | CF₃ |
| 4 | H | CH | Allyl | I | CF₃ |
| 5 | F | CH | Allyl | Br | Br |
| 6 | F | CH | Allyl | Br | I |
| 7 | F | CH | Allyl | Br | CF₃ |
| 8 | F | CH | Allyl | I | CF₃ |
| 9 | CN | CH | Allyl | Br | Br |
| 10 | CN | CH | Allyl | Br | I |
| 11 | CN | CH | Allyl | Br | CF₃ |
| 12 | CN | CH | Allyl | I | CF₃ |
| 13 | CF₃ | CH | Allyl | Br | Br |
| 14 | CF₃ | CH | Allyl | Br | I |
| 15 | CF₃ | CH | Allyl | Br | CF₃ |
| 16 | CF₃ | CH | Allyl | I | CF₃ |
| 17 | F | N | Allyl | Br | Br |
| 18 | F | N | Allyl | Br | I |
| 19 | F | N | Allyl | Br | CF₃ |
| 20 | F | N | Allyl | I | CF₃ |
| 21 | CN | N | Allyl | Br | Br |
| 22 | CN | N | Allyl | Br | I |
| 23 | CN | N | Allyl | Br | CF₃ |
| 24 | CN | N | Allyl | I | CF₃ |
| 25 | CF₃ | N | Allyl | Br | Br |
| 26 | CF₃ | N | Allyl | Br | I |
| 27 | CF₃ | N | Allyl | Br | CF₃ |
| 28 | CF₃ | N | Allyl | I | CF₃ |
| 29 | H | CH | Propargyl | Br | Br |
| 30 | H | CH | Propargyl | Br | I |
| 31 | H | CH | Propargyl | Br | CF₃ |
| 32 | H | CH | Propargyl | I | CF₃ |
| 33 | F | CH | Propargyl | Br | Br |
| 34 | F | CH | Propargyl | Br | I |
| 35 | F | CH | Propargyl | Br | CF₃ |
| 36 | F | CH | Propargyl | I | CF₃ |
| 37 | CN | CH | Propargyl | Br | Br |
| 38 | CN | CH | Propargyl | Br | I |
| 39 | CN | CH | Propargyl | Br | CF₃ |
| 40 | CN | CH | Propargyl | I | CF₃ |
| 41 | CF₃ | CH | Propargyl | Br | Br |
| 42 | CF₃ | CH | Propargyl | Br | I |
| 43 | CF₃ | CH | Propargyl | Br | CF₃ |
| 44 | CF₃ | CH | Propargyl | I | CF₃ |
| 45 | F | N | Propargyl | Br | Br |
| 46 | F | N | Propargyl | Br | I |
| 47 | F | N | Propargyl | Br | CF₃ |
| 48 | F | N | Propargyl | I | CF₃ |
| 49 | CN | N | Propargyl | Br | Br |
| 50 | CN | N | Propargyl | Br | I |
| 51 | CN | N | Propargyl | Br | CF₃ |
| 52 | CN | N | Propargyl | I | CF₃ |
| 53 | CF₃ | N | Propargyl | Br | Br |
| 54 | CF₃ | N | Propargyl | Br | I |
| 55 | CF₃ | N | Propargyl | Br | CF₃ |
| 56 | CF₃ | N | Propargyl | I | CF₃ |

5. The amide compound according to claim 4, wherein the amide compound is selected from: compounds in Table 3;
the compounds in Table 3 have the structure shown in General Formula I, and R₁, X, R₂, R₃, and R₄ are shown in Table 3:
TABLE 3

| Compound | | | | | |
|---|---|---|---|---|---|
| Number | R₁ | X | R₂ | R₃ | R₄ |
| 3 | H | CH | Allyl | Br | CF₃ |
| 4 | H | CH | Allyl | I | CF₃ |
| 7 | F | CH | Allyl | Br | CF₃ |
| 8 | F | CH | Allyl | I | CF₃ |
| 11 | CN | CH | Allyl | Br | CF₃ |
| 12 | CN | CH | Allyl | I | CF₃ |
| 19 | F | N | Allyl | Br | CF₃ |
| 20 | F | N | Ally1 | I | CF₃ |
| 23 | CN | N | Allyl | Br | CF₃ |
| 24 | CN | N | Allyl | I | CF₃ |
| 27 | CF₃ | N | Allyl | Br | CF₃ |
| 28 | CF₃ | N | Allyl | I | CF₃ |
| 31 | H | CH | Propargyl | Br | CF₃ |
| 32 | H | CH | Propargyl | I | CF₃ |
| 35 | F | CH | Propargyl | Br | CF₃ |
| 36 | F | CH | Propargyl | I | CF₃ |
| 39 | CN | CH | Propargyl | Br | CF₃ |
| 40 | CN | CH | Propargyl | I | CF₃ |
| 47 | F | N | Propargyl | Br | CF₃ |
| 48 | F | N | Propargyl | I | CF₃ |
| 51 | CN | N | Propargyl | Br | CF₃ |
| 52 | CN | N | Propargyl | I | CF₃ |
| 55 | CF₃ | N | Propargyl | Br | CF₃ |
| 56 | CF₃ | N | Propargyl | I | CF₃ |

6. A compound being an intermediate for preparing the amide compound according to claim 1, wherein the compound has a structure shown in General Formula II below: 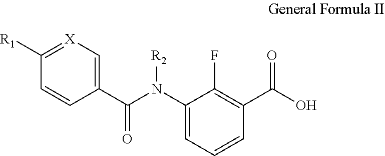
in General Formula II:
R₁ is selected from hydrogen, fluorine, cyano, trifluoromethyl or difluoromethyl;
R₂ is selected from allyl or propargyl; and
X is selected from CH or N.

7. The compound according to claim 6, wherein the compound is selected from: compounds in Table 4;
the compounds in Table 4 have the structure shown in General Formula II, and R₁, X, and R₂ are shown in Table 4:
TABLE 4

| Compound Number | R₁ | X | R₂ |
|---|---|---|---|
| II.1 | H | CH | Allyl |
| II.2 | F | CH | Allyl |
| II.3 | CN | CH | Allyl |
| II.4 | F | N | Allyl |
| II.5 | CN | N | Allyl |
| II.6 | CF₃ | N | Allyl |
| II.7 | H | CH | Propargyl |
| II.8 | F | CH | Propargyl |
| II.9 | CN | CH | Propargyl |
| II.10 | F | N | Propargyl |
| II.11 | CN | N | Propargyl |
| II.12 | CF₃ | N | Propargyl |

8. A compound being an intermediate for preparing the compound according to claim 6, wherein the compound has a structure shown in General formula III below: 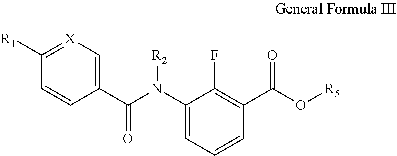
in General Formula III:
R₁ is selected from hydrogen, fluorine, cyano, trifluoromethyl or difluoromethyl;
X is selected from CH or N;
R₂ is selected from allyl or propargyl; and
R₅ is selected from C₁-C₆ alkyl.

9. The compound according to claim 8, wherein the compound is selected from: compounds in Table 5;
the compounds in Table 5 have the structure shown in General Formula III, and R₁, X, R₂, and R₅ are shown in Table 5:
TABLE 5

| Compound | | | | |
|---|---|---|---|---|
| Number | R₁ | X | R₂ | R₅ |
| III.1 | H | CH | Allyl | CH₃ |
| III.2 | F | CH | Allyl | CH₃ |
| III.3 | CN | CH | Allyl | CH₃ |
| III.4 | F | N | Allyl | CH₃ |
| III.5 | CN | N | Allyl | CH₃ |
| III.6 | CF₃ | N | Allyl | CH₃ |
| III.7 | H | CH | Propargyl | CH₃ |
| III.8 | F | CH | Propargyl | CH₃ |
| III.9 | CN | CH | Propargyl | CH₃ |
| III.10 | F | N | Propargyl | CH₃ |
| III.11 | CN | N | Propargyl | CH₃ |
| III.12 | CF₃ | N | Propargyl | CH₃ |
| III.13 | H | CH | Allyl | CH₂CH₃ |
| III.14 | F | CH | Allyl | CH₂CH₃ |
| III.15 | CN | CH | Allyl | CH₂CH₃ |
| III.16 | F | N | Allyl | CH₂CH₃ |
| III.17 | CN | N | Allyl | CH₂CH₃ |
| III.18 | CF₃ | N | Allyl | CH₂CH₃ |
| III.19 | H | CH | Propargyl | CH₂CH₃ |
| III.20 | F | CH | Propargyl | CH₂CH₃ |
| III.21 | CN | CH | Propargyl | CH₂CH₃ |
| III.22 | F | N | Propargyl | CH₂CH₃ |
| III.23 | CN | N | Propargyl | CH₂CH₃ |
| III.24 | CF₃ | N | Propargyl | CH₂CH₃ |

10. A compound being an intermediate for preparing the compound according to claim 8, wherein the compound has a structure shown in General Formula IV below: 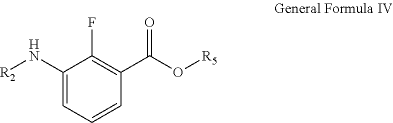
in General Formula IV:
R₂ is selected from allyl or propargyl; and
R₅ is selected from C₁-C₆ alkyl.

11. The compound according to claim 10, wherein the compound is selected from: compounds in Table 6;
the compounds in Table 6 have the structure shown in General formula IV, and R₁ and R₅ are shown in Table 6:
TABLE 6

| Compound Number | R₂ | R₅ |
|---|---|---|
| IV.1 | Allyl | CH₃ |
| IV.2 | Allyl | CH₂CH₃ |
| IV.3 | Allyl | CH₂CH₂CH₃ |
| IV.4 | Ally1 | CH₂CH₂CH₂CH₃ |
| IV.5 | Allyl | CH₂CH₂CH₂CH₂CH₃ |
| IV.6 | Allyl | CH₂CH₂CH₂CH₂CH₂CH₃ |
| IV.7 | Propargyl | CH₃ |
| IV.8 | Propargyl | CH₂CH₃ |
| IV.9 | Propargyl | CH₂CH₂CH₃ |
| IV.10 | Propargyl | CH₂CH₂CH₂CH₃ |
| IV.11 | Propargyl | CH₂CH₂CH₂CH₂CH₃ |
| IV.12 | Propargyl | CH₂CH₂CH₂CH₂CH₂CH₃ |

12. A compound being an intermediate for preparing the amide compound according to claim 1, wherein the compound has a structure shown in General Formula V below: 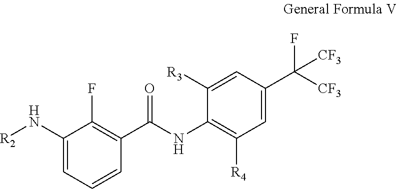
in General Formula V:
R₂ is selected from allyl or propargyl;
R₃ selected from halogen; and
R₄ is selected from halogen, C₁-C₃ haloalkyl, or C₁-C₃ haloalkoxy.

13. The compound according to claim 12, wherein in General Formula V,
R₂ is selected from allyl or propargyl;
R₃ is selected from bromine or iodine; and
R₄ is selected from bromine, iodine, trifluoromethyl or difluoromethoxy.

14. The compound according to claim 13, wherein the compound is selected from: compounds in Table 7;
the compounds in Table 7 have the structure shown in General Formula V, and R₂, R₃ and R₄ are shown in Table 7:
TABLE 7

| Compound Number | R₂ | R₃ | R₄ |
|---|---|---|---|
| V.1 | Allyl | Br | Br |
| V.2 | Allyl | Br | I |
| V.3 | Allyl | Br | CF₃ |
| V.4 | Allyl | I | CF₃ |
| V.5 | Ally1 | Br | OCHF₂ |
| V.6 | Allyl | I | OCHF₂ |
| V.7 | Propargyl | Br | Br |
| V.8 | Propargyl | Br | I |
| V.9 | Propargyl | Br | CF₃ |
| V.10 | Propargyl | I | CF₃ |
| V.11 | Propargyl | Br | OCHF₂ |
| V.12 | Propargyl | I | OCHF₂ |

15. The compound according to claim 14, wherein the compound is selected from: compounds in Table 8;
the compounds in Table 8 have the structure shown in General Formula V, and R₂, R₃ and R₄ are shown in Table 8:
TABLE 8

| Compound Number | R₂ | R₃ | R |
|---|---|---|---|
| V.3 | Allyl | Br | CF₃ |
| V.4 | Allyl | I | CF₃ |
| V.9 | Propargyl | Br | CF₃ |
| V.10 | Propargyl | I | CF₃ |

16. A compound being an intermediate for preparing the amide compound according to claim 1, wherein the compound has a structure shown in General Formula VI below: 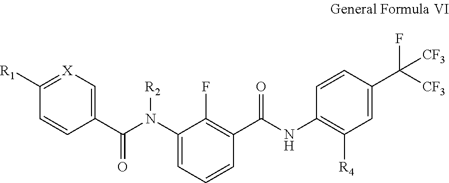
in General Formula VI:
R₁ is selected from hydrogen, fluorine, cyano, trifluoromethyl or difluoromethyl;
R₂ is selected from allyl or propargyl;
R₄ is selected from halogen, C₁-C₃ haloalkyl, or C₁-C₃ haloalkoxy; and
X is selected from CH or N.

17. The compound according to claim 16, wherein in General Formula VI,
R₁ is selected from hydrogen, fluorine, cyano, trifluoromethyl or difluoromethyl;
R₂ is selected from allyl or propargyl;
R₄ is selected from bromine, iodine, trifluoromethyl or difluoromethoxy; and
X is selected from CH or N.

18. The compound according to claim 17, wherein the compound is selected from: compounds in Table 9;
the compounds in Table 9 have the structure shown in General Formula VI, and R₁, X, R₂ and R₄ are shown in Table 9:
TABLE 9

| Compound Number | R₁ | X | R₂ | R4 |
|---|---|---|---|---|
| VI.1 | H | CH | Allyl | CF₃ |
| VI.2 | F | CH | Allyl | CF₃ |
| VI.3 | CN | CH | Allyl | CF₃ |
| VI.4 | F | N | Allyl | CF₃ |
| VI.5 | CN | N | Allyl | CF₃ |
| VI.6 | CF₃ | N | Allyl | CF₃ |
| VI.7 | H | CH | Propargyl | CF₃ |
| VI.8 | F | CH | Propargyl | CF₃ |
| VI.9 | CN | CH | Propargyl | CF₃ |
| VI.10 | F | N | Propargyl | CF₃ |
| VI.11 | CN | N | Propargyl | CF₃ |
| VI.12 | CF₃ | N | Propargyl | CF₃ |
| VI.13 | H | CH | Allyl | OCHF₂ |
| VI.14 | F | CH | Allyl | OCHF₂ |
| VI.15 | CN | CH | Allyl | OCHF₂ |
| VI.16 | F | N | Allyl | OCHF₂ |
| VI.17 | CN | N | Allyl | OCHF₂ |
| VI.18 | CF₃ | N | Allyl | OCHF₂ |
| VI.19 | H | CH | Propargyl | OCHF₂ |
| VI.20 | F | CH | Propargyl | OCHF₂ |
| VI.21 | CN | CH | Propargyl | OCHF₂ |
| VI.22 | F | N | Propargyl | OCHF₂ |
| VI.23 | CN | N | Propargyl | OCHF₂ |
| VI.24 | CF₃ | N | Propargyl | OCHF₂ |

19. The compound according to claim 18, wherein the compound is selected from: compounds in Table 10;
the compounds in Table 10 have the structure shown in General Formula VI, and R₁, X, R₂ and R₄ are shown in Table 10:
TABLE 10

| Compound Number | R₁ | X | R₂ | R₄ |
|---|---|---|---|---|
| VI.1 | H | CH | Allyl | CF₃ |
| VI.2 | F | CH | Allyl | CF₃ |
| VI.3 | CN | CH | Allyl | CF₃ |
| VI.4 | F | N | Allyl | CF₃ |
| VI.5 | CN | N | Allyl | CF₃ |
| VI.6 | CF₃ | N | Allyl | CF₃ |
| VI.7 | H | CH | Propargyl | CF₃ |
| VI.8 | F | CH | Propargyl | CF₃ |
| VI.9 | CN | CH | Propargyl | CF₃ |
| VI.10 | F | N | Propargyl | CF₃ |
| VI.11 | CN | N | Propargyl | CF₃ |
| VI.12 | CF₃ | N | Propargyl | CF₃ |

20. A compound being an intermediate for preparing the compound according to claim 16, wherein the compound has a structure shown in General Formula VII below: 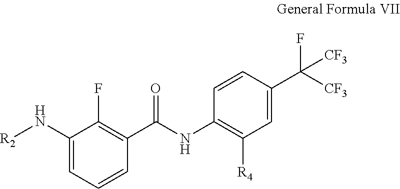
in General Formula VII:
R₂ is selected from allyl or propargyl; and
R₄ is selected from halogen, C₁-C₃ haloalkyl, or C₁-C₃ haloalkoxy.

21. The compound according to claim 20, wherein in General Formula VII,
R₂ is selected from allyl or propargyl; and
R₄ is selected from bromine, iodine, trifluoromethyl or difluoromethoxy.

22. The compound according to claim 21, wherein the compound is selected from: compounds in Table 11;
the compounds in Table 11 have the structure shown in General Formula VII, and R₂ and R₄ are shown in Table 11:
TABLE 11

| Compound Number | R₂ | R₄ |
|---|---|---|
| VII.1 | Allyl | CF₃ |
| VII.2 | Allyl | OCHF₂ |
| VII.3 | Propargyl | CF₃ |
| VII.4 | Propargyl | OCHF₂ |

23. A preparation method for the amide compound according to claim 1, wherein the preparation method comprises: reacting the intermediate compound shown in General Formula VI with a suitable halogenated reagent in a suitable solvent to prepare the compound of General Formula I, and a compound reaction formula is as follows:

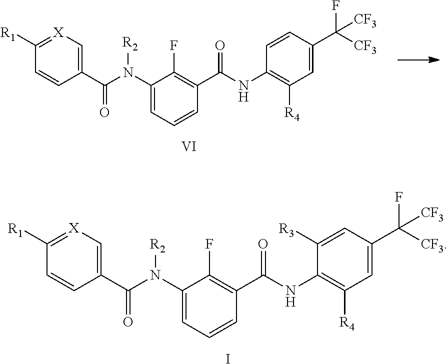

24. The preparation method for the amide compound according to claim 23, wherein the compound of General Formula VI reacts with a suitable alkali or halogenated reagent in a suitable solvent at a temperature from −10° C. to the boiling point of the solvent for 0.5-48 h to prepare the compound of General Formula I.

25. The preparation method for the amide compound according to claim 23, wherein the suitable halogenated reagent is selected from the group consisting of chlorine gas, liquid bromine, iodine, NBS, NCS, NIS, a mixture of hydrogen peroxide and hydrobromic acid, a mixture of hydrogen peroxide and hydroiodic acid, a mixture of sodium hypochlorite and hydrobromic acid, and a mixture of sodium hypochlorite and hydroiodic acid;
the suitable solvent is selected from the group consisting of benzene, toluene, xylene, acetone, methyl ethyl ketone, methyl isobutyl ketone, chloroform, dichloromethane, methyl acetate, ethyl acetate, tetrahydrofuran, dioxane, diethyl ether, 1,2-dimethoxyethane, water, acetonitrile, N,N-dimethylformamide, N,N-dimethylacetamide, N-methylpyrrolidone, dimethyl sulfoxide and mixed solvents of the above solvents; and
the reaction temperature is preferably 0° C.-100° C.; and more preferably 25° C.-80° C.

26. The preparation method for the amide compound according to claim 24, wherein the suitable alkali is selected from the group consisting of trimethylamine, triethylamine, pyridine, DBU, 4-dimethylaminopyridine, N,N-diisopropylmethylamine, N,N-diisopropylethylamine, sodium hydride, potassium hydride, sodium hydroxide, potassium hydroxide, calcium hydroxide, sodium carbonate, potassium carbonate, sodium bicarbonate, potassium bicarbonate, sodium methanol, sodium ethanol, potassium ethanol, potassium tert-butanol and sodium tert-butanol; and preferably, the suitable alkali is selected from the group consisting of sodium hydride, potassium hydride, sodium hydroxide, and potassium hydroxide.

27. An insecticide prepared from the amide compound according to claim 1.

28. The insecticideaccording to claim 27, wherein the insecticide is used to prevent and control one or more of Diamondback moth, Mythimna separata, Spodoptera exigua, Spodoptera litura, Chilo suppressalis, Myzus persicae, thripid, and flea beetle.

29. An insecticide formulation, wherein the insecticide formulation comprises the amide compound according to claim 1 as an active component, and also comprises one or more auxiliary materials; optionally, the amount of the amide compound according to claim 1 in the insecticide formulation is 0.1 to 99% by weight, and further optionally 0.5 to 90% by weight.

30. An insecticide composition, comprising a mixture of the amide compound according to claim 1 and other active compounds, wherein the other active compounds are selected from one or more of an insecticide, a poison bait agent, a disinfectant, an acaricide, a nematicide, a fungicide, a growth regulator, and a herbicide.

31. A method for controlling an agricultural or forestal pest, comprising: applying an effective amount of a material to the pest that needs to be controlled or its growth medium, wherein the material is one or more selected from the group consisting of:
the amide compound according to claim 1;
the insecticide formulation comprising the amide compound according to claim 1; and
the insecticide composition comprising a mixture of the amide compound according to claim 1 and other active compounds.

32. An animal parasite control agent prepared from the amide compound according to claim 1.

33. The control agent according to claim 32, wherein the animal parasite control agent is used to prevent and control one or more of cat flea and American dog tick.

34. An animal parasite control agent, wherein the animal parasite control agent comprises the amide compound according to claim 1 as an active component, and further comprises one or more auxiliary materials; and optionally, the amount of the amide compound according to claim 1 in the animal parasite control agent is 1 to 80% by weight.

35. An animal parasite control agent, comprising a mixture of the amide compound according to claim 1 and other animal parasite control active compounds, wherein the other animal parasite control active compounds are one or more selected from the group consisting of an acaricide, an insecticide, a parasiticide, and an antiplasmodial agent.

36. A method for controlling an animal parasite comprising the following steps: applying an effective amount of a material to the animal parasite that needs to be controlled or its growth medium, wherein the material is one or more selected from the group consisting of the following groups:
the amide compound according to claim 1;
the animal parasite control agent comprising the amide compound according to claim 1; and
the animal parasite control composition comprising the amide compound according to claim 1 and other animal parasite control active compounds.
